(19) United States
(12) Patent Application Publication
(10) Pub. No.: US 2003/0187261 A1
(43) Pub. Date: Oct. 2, 2003

(54) Purine derivatives, process for their preparation and use thereof

(76) Inventors: Libor Havlicek, Praha (CZ); Vladimir Krystof, Ostrava (CZ); Vera Siglerova, Praha (CZ); Rene Lenobel, Sternberk (CZ); Henri Van Onckelen, Antwerp (BE); Zwi Nisan Berneman, Antwerp (BE); Herman Slegers, Pulle-Zandhoven (BE); Edgard Esmans, Antwerp (BE); Miroslav Strnad, Olomouc (CZ); Katrien Vermeulen, Kruibeke (BE)

Correspondence Address: Barbara E Johnson, 700 Koppers Building, 436 Seventh Avenue, Pittaburgh, PA, 15219-1818, US

(21) Appl. No.: 10/169,592

(22) PCT Filed: Jan. 8, 2001
(86) PCT No.: PCT/EP01/00150

(30) Foreign Application Priority Data
Jan. 7, 2000 (EP) 00200070.1

(51) Int. Cl. C07D 473/18; C07D 471/02

(52) U.S. Cl. 544/276; 544/280; 546/118

(57) ABSTRACT

The present invention relates to new purine derivatives and their deaza- and aza-analogues, methods for preparing said derivatives, and to their use in suitable utilities, in particular their use in diagnostics and therapeutic methods. The invention relates in particular to purine derivatives with an inhibitory effect on for example cyclin-dependent kinase proteins (sdks), viruses, and proliferation of haemotapoietic and cancer cells.

The present invention relates to new purine derivatives and their deaza- and aza-analogues, and to their use in suitable utilities, in particular, diagnostic- and therapeutic methods.

The invention relates in particular to purine derivatives with an inhibitor effect with respect to cyclin-dependent kinase proteins (cdks) and also with an inhibitory effect with respect to viruses and immunostimulation.

The use of 2,6,9-trisubstituted purine derivatives as cdk-inhibitor is for example disclosed in WO 97/16452, WO 98/05335, WO/9720842, WO 97/16542, WO98/05335, WO 98/39007, WO 98/49146 and WO 99/07705.

Nucleotide analogues containing phosphonate groups are for example disclosed in U.S. Pat. Nos. 4,659,825; 4,724,233; 5,124,051; 5,302,585; 5,208,221; 5,352,786; 5,356,886; 5,142,051; in EP publication numbers 269,947; 481,214; 630,381; 369,409; 454,427; 618,214; 398,231; 454,427; 468,119; 481,119; 481,214; 434,450 and in WO 95/07920; WO 094/03467, WO96/33200 and WO94/03467. The typical purine base is adenine, 2,6-diaminopurine and guanine. The purine bases may include the aza- and deaza-analogues thereof. 6,9-Substituted and 2,6,9-trisubstituted purines and related analogues are disclosed in WO 96/33200. However, the selectivity and efficiency of these compounds when used for example as anticancer or anti-inflammatory agents has not been satisfactory.

It is the object of this invention to provide anticancer, anti-inflammatory, antiviral, antineuro-degenerative, neurodepressive and immunosuppressive compounds having improved selectivity and efficiency index, i.e. compounds that are less toxic, yet more efficacious than derivatives known heretofore.

This object is achieved by the present invention by providing 2-, 6-, 8-, 9-monosubstituted, 2,6-, 2,9-, 6,8-, 6,9-disubstituted and 2,6,8-2,6,9-, 6,8,9-trisubstituted purine derivatives and related aza-deaza analogues having at least one amine substituted with a catechol group or related group (1,2-dihydroxy-benzene) and the pharmaceutically acceptable salts thereof. The purine derivatives according to the present invention are useful, for example, for inhibiting cdk-activity and also for inhibiting cell proliferation and/or inducing apoptosis.

The present invention also provides methods for, preparing said purine derivatives.

The inventions further relates to the use of the purine derivatives in methods for treatment of the human and animal body.

In addition, the invention provides pharmaceutical compositions comprising as the active ingredient one or more of the purine derivatives, together with at least a pharmaceutically acceptable carrier or diluent.

### SUMMARY OF THE INVENTION

The present invention provides 2-, 6-, 8-, 9-monosubstituted, 2,6-, 2,9-, 6,8-, 6,9-disubstituted and 2,6,8-2,6,9-, 6,8,9-trisubstituted purine derivatives and related aza-deaza analogues represented by general formula I:

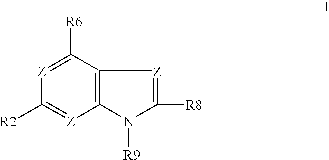

and pharmaceutically acceptable salts thereof, wherein:

Z is N or CH, provided that at most one Z is CH;

R6 is H, halogen, amino, hydroxyl, (substituted) cycloalkyl, (substituted) cycloalkyl alkyl, (substituted) cycloheteroalkyl, (substituted) arylalkyl, (substituted) heteroalkyl, (substituted) heteroarylalkyl or R6′-X wherein

X is —NH—, —N(alkyl)-, —O— or —S—; and

R6′ is (substituted) cycloalkyl, (substituted) aryl, (substituted) heterocycle, (substituted) heteroaryl, (substituted) arylalkyl, (substituted) cycloheteroalkyl, (substituted) heteroarylalkyl, (substituted) heteroalkyl, (substituted) cycloalkyl alkyl or (substituted) cycloheteroalkyl alkyl;

R8 is H, halogen, hydroxyl, amino, carboxyl, cyano, nitro, amido, sulfo, sulfamido, carbamino, (substituted) alkyl, (substituted) acyl, (substituted) cycloalkyl, (substituted) cycloheteralkyl, (substituted) arylalkyl, (substituted) heteroalkyl, (substituted) heteroaryl, (substituted) heterocycle, (substituted) heteroarylalkyl, (substituted) cycloalkyl alkyl, (substituted) aryl, (substituted) cycloheteroalkyl alkyl or R8′-X, wherein

X is is —NH—, —N(alkyl)-, —O— or —S—; and

R8′ is H, (substituted) alkyl, (substituted) acyl, amido, sulfo, (substituted) cycloalkyl, (substituted) aryl, (substituted) heterocycle, (substituted) heteroaryl, (substituted) arylalkyl, (substituted) cycloheteroalkyl, (substituted) heteroarylalkyl, (substituted) heteroalkyl, (substituted) cycloalkyl alkyl or (substitited) cycloheteroalkyl alkyl;

R2 is H, halogen, amido, carbamino, carboxyl, sulfamido, (subsituted) alkyl, (substituted) cycloalkyl, (substituted) cycloalkyl alkyl, (substituted) arylalkyl, (substituted) heteroalkyl, (substituted) heteroarylalkyl, (substituted) cycloheteroalkyl alkyl or R2′-X wherein

X is —NH—, —N(alkyl)-, —O— or —S—;

R2′ is H, (substituted) alkyl, (substituted) acyl, amido, sulfo, carbamino, (substituted) cycloalkyl, (substituted) aryl, (substituted) heterocycle, (substituted) heteroaryl, (substituted) arylalkyl, (substituted) cycloheteroalkyl, (substituted) heteroarylalkyl, (substituted) heteroalkyl, (substituted) cycloalkyl alkyl or (substituted) cycloheteroalkyl alkyl; and

R9 is H, (substituted) alkyl, (substituted) acyl, carboxyl, amido, sulfo, sulfamido, carbamino, (substituted) cycloalkyl, (substituted) cycloalkyl alkyl, (substituted) cycloheteroalkyl alkyl, (substituted) cycloheteroalkyl, (substituted) aryl, (substituted) heterocycle, (substituted) heteroaryl, (substituted) arylalkyl (substituted) heteroarylalkyl, (substituted) heteroalkyl;

or —(CH₂)ₙ—R9′, wherein

n=1-2; and

R9′ is —X(CH₂)ₘY; wherein

X is —O—, —S—, —NH— or —N(alkyl)-;

m=1-2;

Y is carboxyl, amido, sulfo, sulfamido, hydroxy, alkoxy, mercapto, alkylmercapto, amino, alkylamino, carbamino —PO(OH)₂, —PO(Oalkyl)₂, —PO(NHalkyl)₂, —PO(Oalkyl)(NHalkyl), —PO(OH)(Oalkyl), —PO(OH)(NHalkyl); or

R9 is —(CH₂CHD)—R9′, wherein

R9′ is —X(CH₂)ₘY; wherein

X is —O—, —S—, —NH—, —N(alkyl)-;

m=1-2;

Y is carboxyl, amido, sulfo, sulfamido, hydroxy, alkoxy, mercapto, alkylmercapto, amino, alkylamino, carbamino, —PO(OH)₂, PO(Oalkyl)₂, —PO(NHalkyl), —PO(OH)(Oalkyl), —PO(OH)(NHalkyl), PO(Oalkyl)(Nalkyl); and

D is (substituted) alkyl;

wherein at least one of R2, R6, R8 or R9 is an amine substituted with a catechol group or related group.

The purine derivatives of the invention preferably are used as an inhibitor of cyclin-dependent kinase proteins (cdks), as an antiviral, antimitotic, antiproliferative, immunomodulating, immune-suppressive, anti-inflammatory and/or antitumor agent. In addition, the purine derivatives of the invention can be used as modulator of β-adrenergic and/or purinergic receptors, as an inhibitor of proliferation of hematopoietic cells and cancer cells, and/or an inducer of apoptosis in cancer cells.

In a preferred embodiment the invention provides purine derivatives for use in the treatment of the human or animal body.

In another preferred embodiment the invention provides a pharmaceutical composition comprising one or more purine derivatives of the invention and a pharmaceutically acceptable carrier or diluent.

The purine derivatives according to the present invention are furthermore preferably used for several other applications, such as for the preparation of affinity absorption matrices.

### DETAILED DESCRIPTION OF THE INVENTION

As used herein, unless modified by the immediate context:

“Halogen” refers to fluorine, bromine, chlorine and iodine atoms.

“Hydroxyl” refers to —OH.

“Mercapto” refers to —SH.

“Alkyl” refers to a branched or unbranched C₁-C₆ chain which may be saturated or unsaturated, such as for example methyl, propyl, isopropyl, tert-butyl, allyl, vinyl, ethinyl, propargyl, or hexen-2-yl.

“Substituted alkyl” refers to an alkyl as defined above, including one or more substituents such as hydroxyl, mercapto, alkylmercapto, halogen, alkoxy, amino, amido, carboxyl, sulfo, acyl and the like. These groups may be attached to any carbon atom of the alkyl moiety.

“Alkoxy” refers to —OR, wherein R is (substituted) alkyl, (substituted) aryl (substituted) arylalkyl, (substituted) cycloalkyl, (substituted) cycloheteroalkyl as defined.

“Alkylmercapto” relates to —SR, wherein R is as defined for “alkoxy”.

“Sulfo” refers to —SO₃R, wherein R is H, alkyl or substituted alkyl.

“Sulfamido” refers to the group —NHSO₃R, wherein R is H, alkyl or substituted alkyl.

“Acyl” refers to —C(O)R, wherein R is hydrogen, (substituted) alkyl, (substituted) aryl, (substituted) arylalkyl, (substituted) cycloalkyl as defined herein.

“Aryloxy” refers to groups —OAr, wherein Ar is (substituted aryl), or (substituted) heteroaryl group as defined herein.

“Alkylamino” refers to the group —NRR′, wherein R and R′ may independently be hydrogen, (substituted) alkyl, (substituted) aryl, or (substituted) heteroaryl as defined herein.

“Amido” refers to the group —C(O)NRR′, wherein R and R′ may independently be hydrogen, (substituted) alkyl, (substituted) aryl, or (substituted) heteroaryl as defined herein.

“Carboxyl” refers to the group —C(O)OR, wherein R is hydrogen, (substituted) alkyl, (substituted) aryl, or (substituted) heteroaryl as defined herein.

“Carbamino” refers to the group —NHCOR, wherein R is hydrogen, (substituted) alkyl, heterocycle, (substituted) aryl, or (substituted) heteroaryl as defined herein.

“Aryl” or “Ar” refers to an aromatic carbocyclic group having at least one aromatic ring (e.g., phenyl or biphenyl) or multiple condensed rings in which at least one ring is aromatic (e.g., 1,2,3,4-tetrahydronaphthyl, naphthyl, anthryl, or phenanthryl).

“Substituted aryl” refers to aryl as defined above, optionally substituted with one or more functional groups such as halogen, alkyl, hydroxyl, amino, mercapto, alkoxy, alkylmercapto, alkylamino, amido, carboxyl, nitro, sulfo and the like.

“Heterocycle” refers to an unsaturated or aromatic carbocyclic group having at least one heteroatom, such as N, O or S, within the ring; the ring can be single condensed (e.g. pyranyl, pyridyl or furyl) or multiple condensed (e.g., quinazolinyl, purinyl, quinolinyl or benzofuranyl), which can optionally be substituted with, e.g., halogen, alkyl, alkoxy, alkylmercapto, alkylamino, amido, carboxyl, hydroxyl, nitro, mercapto, sulfo and the like.

“Heteroaryl” refers to a heterocycle in which at least one heterocyclic ring is aromatic.

“Substituted heteroaryl” refers to a heterocycle which optionally is monosubstituted or polysubstituted with one or more functional groups, e.g., halogen, alkyl, alkoxy, alkylthio, alkylamino, amido, carboxyl, hydroxyl, nitro, mercapto, sulfo and the like.

“(Substituted) arylalkyl” refers to the group —R—Ar herein Ar is an aryl group and R is alkyl or substituted alkyl group. The aryl groups are optionally substituted with, e.g., halogen, alkyl, hydroxyl, alkoxy, alkylmercapto, alkylamino, amido, carboxyl, hydroxy, aryl, nitro, mercapto, sulfo and the like.

“(Substituted) heteroalkyl” refers to the group —R-Het, wherein Het is a heterocycle group and R is an alkyl group. The heteroalkyl groups are optionally substituted with e.g., halogen, alkyl, alkoxy, alkylthio, alkylamino, amido, carboxy, alkoxycarbonyl, aryl, aryloxy, nitro, thiol, sulfonyl and the like.

“(Substituted) heteroarylalkyl” refers to the group —R-HetAr wherein HetAr is a heteroaryl group and R is alkyl or substituted alkyl. Heteroarylalkyl groups are optionally substituted with, e.g., halogen, alkyl, substituted alkyl, alkoxy, alkylmercapto, nitro, thiol, sulfo and the like.

“Cycloalkyl” refers to a divalent cyclic or polycyclic alkyl group containing 3 to 15 carbon atoms.

“Substituted cycloalkyl” refers to a cycloalkyl group comprising one or more substituents such as, e.g., halogen, alkyl, substituted alkyl, alkoxy, alkylmercapto, aryl, nitro, mercapto, sulfo and the like.

“Cycloheteroalkyl” refers to a cycloalkyl group wherein one or more of the ring carbon atoms is replaced with a heteroatom (e.g., N, O, S or P).

“Substituted cycloheteroalkyl” refers to a cycloheteroalkyl group as defined above, which contains one or more substituents, such as halogen, alkyl, alkoxy, alkylmercapto, alkylamino, amido, carboxyl, hydroxy, nitro, mercapto, sulfo and the like.

“(Substituted) cycloalkyl alkyl” refers to the group —R-cycloalkyl wherein cycloalkyl is a cycloalkyl group and R is an alkyl or substituted alkyl. The cycloalkyl group can optionally be substituted with e.g., halogen, alkyl, alkoxy, alkylmercapto, alkylamino, amido, carboxyl, hydroxy, nitro, mercapto, sulfo and the like.

“(Substituted) cycloheteroalkyl alkyl” refers to —R-cycloheteroalkyl wherein R is alkyl or substituted alkyl. The cycloheteroalkyl group can optionally be substituted with e.g. halogen, alkyl, alkoxy, alkylmercapto, alkylamino, amido, carboxyl, hydroxy, nitro, mercapto, sulfo and the like.

“An amine substituted with a catechol group or related group” refers to secondary and tertiary amines containing at least one dihydroxyaryl or dihydroxyarylalkyl group”.

The present invention provides purine derivatives and related aza-deaza analogues represented by general formula I:

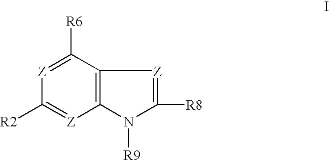

and pharmaceutically acceptable salts thereof, wherein:

Z is N or CH, provided that at most one Z is CH;

R6 is H

halogen;

amino;

hydroxyl;

cycloalkyl, such as cyclopropyl, cyclopentyl, cyclohexyl or adamantyl, optionally substituted with at least one halogen, amino, hydroxy, cyano, nitro, mercapto, alkoxy, alkylamino, dialkylamino, alkylmercapto, carboxyl, amido, sulfo, sulfamido or carbamino;

cycloalkyl alkyl (—R-cycloalkyl), wherein R is a lower alkyl, such as methyl, ethyl, propyl, isopropyl, vinyl, ethinyl, propenyl, or propinyl, optionally substituted with at least one halogen, amino, hydroxy, mercapto, alkoxy, alkylamino, dialkylamino, alkylmercapto, carboxyl, amido, sulfo, sulfamido or carbamino;

arylalkyl (—R—Ar), wherein R is a a lower alkyl, such as methyl, ethyl, propyl, isopropyl, vinyl, propinyl, propenyl, or ethinyl, and Ar is phenyl, biphenyl, tetrahydronaphthyl, naphthyl, anthryl, indenyl, or fenanthryl, optionally substituted with one or more groups as defined for cycloalkyl;

heteroalkyl (—R-Het), wherein R is a lower alkyl, such as methyl, ethyl, propyl, isopropyl, vinyl, propinyl, propenyl or ethinyl, and Het is thienyl, furyl, pyranyl, pyrrolyl, imidazolyl, pyrazolyl, pyridyl, pyrazolinyl, pyrimidinyl, pyridazinyl, isothiazolyl, or isoxazolyl, optionally substituted with substituents defined for cycloalkyl;

heteroaryl alkyl (—R-HetAr), wherein R is a lower alkyl, such as methyl, ethyl, propyl, isopropyl, vinyl, propinyl, propenyl, ethinyl, and wherein HetAr is benzothienyl, naphthothienyl, benzofuranyl, chromenyl, indolyl, isoindolyl, indazolyl, quinolyl, isoquinolyl, phtalazinyl, quinaxalinyl, cinnolinyl, or quinazolinyl, optionally substituted with substituents as defined for cycloalkyl;

cycloheteroalkyl (—R-cycloheteroalkyl), wherein R is a lower alkyl, such as methyl, ethyl, propyl, isopropyl, vinyl, propinyl, propenyl or ethinyl and wherein cycloheteroalkyl is pyrrolidinyl, piperidinyl, morfolinyl, imidazolidinyl, imidazolinyl or quinuclidinyl and the cycloheteroalkyl ring optionally is substituted with at least one hydroxyl, amino, mercapto, carboxyl, amido or sulfo substituent; or

R6′-X, wherein

X is —NH—, —O—, —S— or —N(substituted arylalkyl)-, such as di- and tri-substituted benzyl, substituted with at least one halogen, cyano, nitro, mercapto, alkoxy, alkylamino, dialkylamino, alkylmercapto, carboxyl, amido, sulfo, sulfamido or carbamino, or α-(aminomethyl)-di- and tri-substituted benzyl, substituted by same substituents as defined for benzyl; and

R6′ is H

acyl (—C(O)R), wherein R is cycloalkyl, cycloalkyl alkyl, aryl, heterocycle, heteroalkyl, heteroaryl, arylakyl, cycloheteroalkyl, cycloheteroalkyl alkyl or heteroarylalkyl, optionally substituted by 1 to 4 substituents, such as halogen, amino, hydroxyl, mercapto, alkoxy, alkylmercapto, alkylamino, carboxyl, amido, sulfo, sulfamido or carbamino;

amido (—C(O)NRR′), wherein R and R′ can independently be H, C₁-C₇ cycloalkyl, cycloalkyl alkyl, aryl, heterocycle, heteroalkyl, heteroaryl, arylalkyl, cycloheteroalkyl, cycloheteroalkyl alkyl or heteroarylalkyl, and R and R′ optionally are substituted by suitable substituents such as benzyl and phenyl;

sulfo (—SO₃R), wherein R is H, C₁-C₆ cycloalkyl, cycloalkyl alkyl, aryl, heterocycle, heteroalkyl, heteroaryl, arylalkyl, cycloheteroalkyl, cycloheteroalkyl alkyl or heteroarylalkyl, optionally substituted by 1 to 4 substituents, such as halogen, amino, hydroxyl, mercapto, carboxyl, amido or carbamino;

carbamino (—NHC(O)R), wherein R is cycloalkyl, cycloalkyl alkyl, aryl, heterocycle, heteroalkyl, heteroaryl, arylakyl, cycloheteroalkyl, cycloheteroalkyl alkyl or heteroarylalkyl, optionally substituted by 1 to 4 substituents, such as halogen, amino, hydroxyl, mercapto, carboxyl, amido or carbamino;

cycloalkyl, optionally substituted by 1 to 4 substituents, such as halogen, such as chloro or fluoro), amino, hydroxyl, mercapto, alkoxy, alkylamino, dialkylamino, alkylmercapto, carboxyl, amido, sulfo, sulfamido, carbamino, nitro or cyano;

cycloalkyl alkyl —R(cycloalkyl), wherein R is a branched or linear, saturated or unsaturated lower alkyl, such as methyl, ethyl, propyl, isopropyl, allyl, propargyl, isopentenyl or isobutenyl, and cycloalkyl is as defined for (substituted) cycloalkyl, and wherein alkyl as well as cycloalkyl are optionally substituted by 1 to 4 substituents, such as halogen, amino, hydroxyl, mercapto, carboxyl, amido or carbamino;

aryl, such as phenyl, biphenyl, naphthyl, tetrahydronaphthyl, fluorenyl, indenyl or fenanthrenyl, optionally substituted by 1 to 4 substituents such as those defined for substituted cycloalkyl, such as chloro, fluoro, hydroxyl, amino, carboxyl or amido;

heterocycle, such as thienyl, furyl, pyranyl, pyrrolyl, imidazolyl, pyrazolyl, pyridyl, pyrazinyl, pyrimidinyl, pyridazinyl, isothiazolyl or isoxazyl, optionally substituted by 1 to 4 substituents such as defined for cycloalkyl;

heteroalkyl (—R-Het), wherein R is a lower alkyl such as methyl, ethyl, propyl, isopropyl, vinyl, propinyl, propenyl or ethinyl, and Het is as described for the heterocycle, which optionally is substituted by 1 to 4 substituents as defined for substituted cycloalkyl;

heteroaryl (—R-HetAr), wherein R is a lower alkyl such as methyl, ethyl, propyl, isopropyl, vinyl, propinyl, or propenyl and HetAr is benzothienyl, naphthothienyl, benzofuranyl, chromenyl, indolyl, isoindolyl, indazolyl, quinolinyl, isoquinolinyl, phtalazinyl, quinaxalinyl, cinnolinyl or quinazolinyl, and wherein the heteroaryl ring optionally is substituted by 1 to 4 substituents as defined for substituted cycloalkyl;

arylalkyl (—RAr), wherein R is a branched or linear, saturated or unsaturated C₁-C₆ lower alkyl such as methyl, ethyl, propyl, isopropyl, vinyl, propinyl, or propenyl, and the alkyl as well as aryl ring(s) optionally are substituted by 1 to 4 independent substituents as defined for substituted cycloalkyl;

cycloheteroalkyl, such as piperidinyl, piperazinyl, morfolinyl, pyrrolidinyl or imidazolidinyl, and wherein the cycloheteralkyl ring optionally is substituted by 1 to 4 substituents such as defined for substituted cycloalkyl;

cycloheteroalkyl alkyl (—R(cycloheteroalkyl), wherein R is as defined for arylalkyl, and alkyl as well as cycloheteroalkyl ring optionally is substituted with 1 to 4 groups as defined for cycloalkyl;

heteroarylalkyl (—R-HetAr), wherein R is a branched or linear, saturated or unsaturated lower alkyl such as methyl, ethyl, propyl, isopropyl, vinyl, propinyl, propenyl, allyl, propargyl or isopentenyl and HetAr is benzothienyl, benzofuranyl, chromenyl, indolyl, isoindolyl, indazolyl, quinolinyl, phthalazinyl, quinoxalinyl, quinazolinyl, carbazolyl, acridinyl, indolinyl or isoindolinyl, and R and HetAr optionally independently are substituted by alkyl, substituted alkyl, halogen, hydroxyl, amino, mercapto, carboxyl or amido;

R2 is H

halogen

C₁-C₆ branched or linear, saturated or unsaturated lower alkyl such as methyl, ethyl, propyl, isopropyl, butyl, isobutyl, vinyl, allyl, ethinyl, propenyl, propinyl or isopenten-2-yl, optionally substituted with halogen, amino, hydroxy, mercapto, alkoxy, alkylamino, dialkylamino, alkylmercapto, carboxyl, amido, sulfo, sulfamido or carbamino;

C₃-C₁₅ cycloalkyl, such as cyclopropyl, cyclopentyl, cyclohexyl, or adamantyl;

cycloalkyl, optionally substituted with halogen, amino, hydroxy, mercapto, alkoxy, alkylamino, dialkylamino, alkylmercapto, carboxyl, amido, sulfo, sulfamido or carbamino;

cycloalkyl alkyl (—R-cycloalkyl), wherein R is a lower alkyl such as methyl, ethyl, propyl, isopropyl, vinyl, ethinyl, propenyl or propinyl;

arylalkyl (—R—Ar), wherein R is a lower alkyl such as methyl, ethyl, propyl, isopropyl, vinyl, propinyl, propenyl or ethinyl, and wherein Ar is phenyl, biphenyl, tetrahydronaphthyl, naphthyl, anthryl, indenyl or fenanthryl, optionally substituted with the groups as defined for cycloalkyl;

heteroalkyl (—R-Het), wherein R is a lower alkyl such as methyl, ethyl, propyl, isopropyl, vinyl, propinyl, propenyl or ethinyl, and Het is thienyl, furyl, pyranyl, pyrrolyl, imidazolyl, pyrazolyl, pyridyl, pyrazolinyl, pyrimidinyl, pyridazinyl, isothiazolyl or isoxazolyl, optionally substituted with substituents as defined for cycloalkyl;

heteroaryl alkyl (—R-HetAr), wherein R is a lower alkyl, such as methyl, ethyl, propyl, isopropyl, vinyl, propinyl, propenyl, or ethinyl, and HetAr is benzothienyl, naphthothienyl, benzofuranyl, chromenyl, indolyl, isoindolyl, indazolyl, quinolinyl, isoquinolinyl, phtalazinyl, quinaxalinyl, cinnolinyl or quinazolinyl; —cycloheteroalkyl (—R-cycloheteroalkyl), wherein R is a lower alkyl such as methyl, ethyl, propyl, isopropyl, vinyl, propinyl, propenyl or ethinyl, and wherein cycloheteroalkyl is pyrrolidinyl, piperidinyl, morfolinyl, imidazolidinyl, imidazolinyl or quinuclidinyl, optionally substituted with hydroxyl, amino, mercapto, carboxyl, amido or sulfo substituents; or R2′-X, wherein

X is —NH—, —O—, —S— or —N(alkyl)-, wherein alkyl is methyl, ethyl, propyl, isopropyl, vinyl, ethinyl, allyl, propargyl or isopentenyl; and

R2′ is

H

C₁-C₆ branched or linear, saturated or unsaturated alkyl, such as methyl, ethyl, isopropyl, butyl, isobutyl, vinyl, allyl, propenyl, propargyl, propinyl, isopentenyl, or isobutenyl, optionally substituted by 1 to 3 substituents, such as halogen, amino, hydroxyl, mercapto, alkoxy, alkylmercapto, alkylamino, carboxyl, amido, sulfo, sulfamido-or carbamino;

acyl (—C(O)R), wherein R is a branched or linear, saturated or unsaturated lower alkyl such as methyl, ethyl, propyl, isopropyl, butyl, isobutyl, vinyl, allyl, propenyl, propargyl, propinyl, isopentenyl or isobutenyl, optionally substituted by 1 to 3 substituents, such as halogen, amino, hydroxyl, mercapto, alkoxy, alkylmercapto, alkylamino, carboxyl, amido, sulfo, sulfamido or carbamino;

amido (—C(O)NRR′), wherein R and R′ independently are H, C₁-C₆ branched or linear, saturated or unsaturated alkyl, and wherein R or R′ optionally are substituted by suitable substituents such as methyl, ethyl, propyl, isopropyl, butyl, isobutyl, vinyl, allyl, propenyl or propargyl;

sulfo (—SO₃R), wherein R is H, or a branched or linear, saturated or unsaturated C₁-C₆ alkyl, optionally substituted by halogen, amino, hydroxyl, mercapto, carboxyl, amido or carbamino;

carbamino (—NHC(O)R), wherein R is a branched or linear, saturated or unsaturated alkyl such as methyl, ethyl, propyl, isopropyl, allyl, propargyl, isopentenyl or isobutenyl, or R is hydroxyl, amino, alkoxy or alkylamino, and wherein R optionally is substituted with halogen, amino, hydroxyl, mercapto, carboxyl or amido;

C₃-C₁ cycloalkyl, such as cyclopropyl, cyclopentyl or cyclohexyl;

cycloalkyl, optionally substitued with 1 to 3 independent substituents, such as halogen (such as chloro or fluoro), amino, hydroxyl, mercapto, alkoxy (such as methoxy), alkylamino, dialkylamino, alkylmercapto, carboxyl, amido, sulfo, sulfamido, carbamino, nitro or cyano;

cycloalkyl alkyl (—R(cycloalkyl)), wherein R is a branched or linear, saturated or unsaturated lower alkyl such as methyl, ethyl, propyl, isopropyl, allyl, propargyl, isopentenyl or isobutenyl, and cycloalkyl is as defined for cycloalkyl and substituted cycloalkyl;

aryl, such as phenyl, biphenyl, naphthyl, tetrahydronaphthyl, fluorenyl, indenyl or fenanthrenyl, optionally substituted by 1 to 3 substituents such as defined for substituted cycloalkyl;

heterocycle such as thienyl, furyl, pyranyl, pyrrolyl, imidazolyl, pyrazolyl, pyridyl, pyrazinyl, pyrimidinyl, pyridazinyl, isothiazolyl or isoxazyl, optionally substituted by 1 to 2 substituents, such as those defined for substituted cycloalkyl;

heteroalkyl (—R-Het), wherein R is a lower alkyl such as methyl, ethyl, propyl, isopropyl, vinyl, propinyl, propenyl or ethinyl, and Het is as defined for the heterocycle group, optionally substituted by 1 to 2 substituents as defined for substituted cycloalkyl;

heteroaryl (—R-HetAr), wherein R is methyl, ethyl, propyl, isopropyl, vinyl, propinyl or propenyl, and HetAr is benzothienyl, naphthothienyl, benzofuranyl, chromenyl, indolyl, isoindolyl, indazolyl, quinolinyl, isoquinolinyl, phtalazinyl, quinaxalinyl, cinnolinyl or quinazolinyl;

arylalkyl (—RAr), wherein R is a branched or linear, saturated or unsaturated C₁-C₆ lower alkyl such as methyl, ethyl, propyl, isopropyl, vinyl, propinyl or propenyl, and the aryl ring(s) optionally are substituted by 1 to 3 independent substituents as defined for substituted cycloalkyl;

cycloheteroalkyl such as piperidinyl, piperazinyl, morfolinyl, pyrrolidinyl or imidazolidinyl, optionally substituted by 1 to 2 substituents such as those defined for substituted cycloalkyl;

cycloheteroalkyl alkyl (—R(cycloheteroalkyl)), wherein R is as defined for arylalkyl, and the cycloheteroalkyl ring optionally is substituted with 1 to 2 groups as defined for cycloalkyl;

heteroarylalkyl (—R-HetAr_, wherein R is a branched or linear, saturated or unsaturated lower alkyl, such as methyl, ethyl, propyl, isopropyl, vinyl, propinyl, propenyl, allyl, propargyl or isopentenyl, and HetAr is benzothienyl, benzofuranyl, chromenyl, indolyl, isoindolyl, indazolyl, quinolinyl, phthalazinyl, quinoxalinyl, quinazolinyl, carbazolyl, acridinyl, indolinyl or isoindolinyl, and wherein R and HetAr optionally independently are substituted by halogen, hydroxyl, amino, mercapto, carboxyl or amido;

R8 is H, halogen, hydroxyl, amino, carboxyl, cyano, nitro, amido, sulfo, sulfamido, carbamino; or

(substituted) alkyl, acyl, (substituted) cycloalkyl, (substituted) cycloheteroalkyl, cycloalkyl alkyl, (substituted) aryl, arylakyl, heterocycle, (substituted) heteroaryl, heteroalkyl or heteroarylalkyl, wherein these groups are as defined for R2; or R8′-X, wherein

X is —NH—, —O—, —S— or —N(alkyl)-, wherein alkyl is C₁-C₆ alkyl, methyl, ethyl, propyl, isopropyl, vinyl, allyl or propargyl; and

R8′ is (substituted) alkyl, acyl, amido, (substituted) cycloalkyl, (substituted) cycloheteroalkyl, cycloalkyl alkyl, (substituted) aryl, arylakyl, heterocycle, (substituted) heteroaryl, heteroalkyl or heteroarylalkyl, wherein these groups are as defined for R2′; and

R9 is H, (substituted) alkyl, acyl, amido, carboxyl, sulfo, carbamino, (substituted) cycloalkyl, (substituted) cycloheteroalkyl, cycloalkyl alkyl, (substituted) aryl, arylalkyl, heterocycle, (substituted) heteroaryl, heteroalkyl or heteroarylalkyl, and wherein these groups are as defined for R2, or (CH₂)ₙ—R9′, wherein

n=1-2; and

R9′ is —X(CH₂)ₘY; wherein

X is —O—, —S—, —NH— or —N(alkyl)-, wherein alkyl is a linear or branched, saturated or unsaturated C₁-C₆ alkyl, such as methyl, ethyl, propyl, isopropyl, vinyl, allyl or propargyl;

m=1-2; and

Y is hydroxy, mercapto, amino, alkoxy, alkylmercapto, alkylamino, carboxyl, sulfo, sulfamido, carbamino, —PO(OH) 2, —PO(Oalkyl)(OH), —PO(Oalkyl)₂, —PO(Oalkyl)(NHalkyl), —PO(NHalkyl)₂, —PO(NHalkyl)(OH);

or (CH₂CHD)-R9′, wherein

R9′ is —X(CH₂)ₘY; wherein

X is —O—, —S—, —NH— or —N(alkyl)-, wherein alkyl is a linear or branched, saturated or unsaturated C₁-C₆ alkyl, such as methyl, ethyl, propyl, isopropyl, vinyl, allyl or propargyl;

m=1-2;

Y is hydroxy, mercapto, amino, alkoxy, alkylmercapto, alkylamino, carboxyl, sulfo, sulfamido, carbamino, —PO(OH) 2, —PO(Oalkyl)(OH), —PO(Oalkyl)₂, —PO(Oalkyl)(NHalkyl), —PO(NHalkyl)₂, or PO(NHalkyl)(OH); and

D is a lower alkyl, optionally substituted with Y.

The invention further provides purine derivatives represented by general formula II or III:

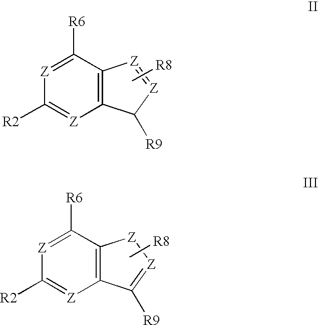

and pharmaceutically acceptable salts thereof, wherein

Z is N, NH or CH, provided that the heterocyclic structures II and III contain 3 or 4 N atoms;

R2, R6 and R9 are as defined in claim 1 or 2; and

R8 is halogen, amino, hydroxyl, mercapto, amido, acyl, (substituted) alkyl, carboxyl, sulfo, sulfamido, carbamino, (substituted) cycloalkyl, (substituted) aryl, heterocycle, (substituted) heteroaryl, (substituted) arylalkyl, (substituted) cycloheteroalkyl, (substituted)heteroalkyl, (substituted) heteroarylalkyl, (substituted) cycloalkyl alkyl or (substituted) cycloheteroalkyl alkyl; or R8′-X, wherein

X is —O—, —S—, —NH— or —N(alkyl)-, and

R8′ is (substituted) alkyl, (substituted) cycloalkyl, (substituted) aryl, heterocycle, (substituted) heteroaryl, arylalkyl, (substituted) cycloheteroalkyl, heteroalkyl, heteroarylalkyl, cycloalkyl alkyl or cycloheteroalkyl alkyl;

wherein:

R8 is absent if both Z in the five-membered ring in formula II are N;

R8 is attached to any Z of the five membered ring in formula III if both Z of that ring are N; or

R8 is attached to one particular Z of the five-membered ring in any of the formulas II and III if that particular Z is CH or CH₂.

In an advantageous embodiment the present invention provides purine derivatives wherein R6=H and R2, R8 and R9 are as defined above.

In another preferred embodiment the invention provides purine derivatives wherein R2=H and R6, R8 and R9 are as defined above.

In yet another embodimnet the invention provides purine derivatives represented by formula IV

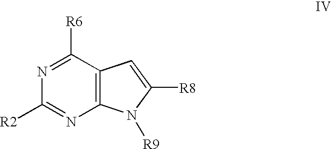

wherein R8=H, and R2, R6, and R9 are as defined above.

In another preferred embodiment the invention provides purine derivatives as represented by formula IV wherein R6=H and R2, R8 and R9 are as defined above.

In another preferred embodiment the invention provides purine derivatives as represented by formula IV wherein R2=H and R6, R8 and R9 are as defined above.

In another preferred embodiment the invention provides purine derivatives as represented by formula v

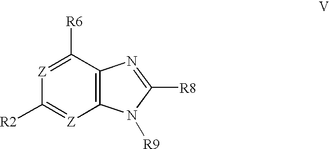

wherein R8=H, and R2, R6 and R9 are as defined above.

In another preferred embodiment the invention provides purine derivatives as represented by formula v wherein R6=H and R2, R8 and R9 are as defined above.

In another preferred embodiment the invention provides purine derivatives as represented by formula V wherein R2=H and R6, R8 and R9 are as defined above.

In another preferred embodiment the invention provides purine derivatives as represented by formula VI

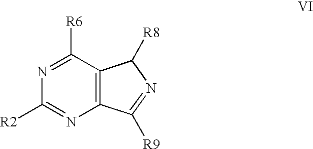

wherein R8H, and R2, R6 and R9 are as defined above.

In another preferred embodiment the invention provides purine derivatives as represented by formula VI wherein R6=H and R2, R8 and R9 are as defined above.

In another preferred embodiment the invention provides purine derivatives as represented by formula VI wherein R2=H and R6, R8 and R9 are as defined above.

In another preferred embodiment the invention provides purine derivatives as represented by formula VII

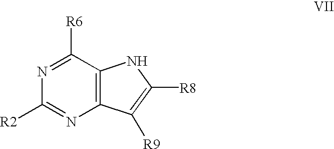

wherein R8=H, and R2, R6 and R9 are as defined above.

In another preferred embodiment the invention provides purine derivatives as represented by formula VII wherein R6=H and R2, R8 and R9 are as defined above.

In another preferred embodiment the invention provides purine derivatives as represented by formula VII wherein R2=H and R6, R8 and R9 are as defined above.

In another preferred embodiment the invention provides purine derivatives as represented by formula VIII

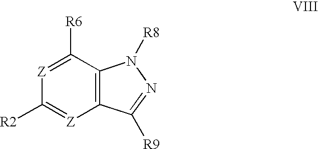

wherein R8=H, and R2, R6 and R9 are as defined above.

In another preferred embodiment the invention provides purine derivatives as represented by formula VIII wherein R6=H and R2, R8 and R9 are as defined above.

In another preferred embodiment the invention provides purine derivatives as represented by formula VIII wherein R2=H and R6, R8 and R9 are as defined above.

In another preferred embodiment the invention provides purine derivatives as represented by formula IX

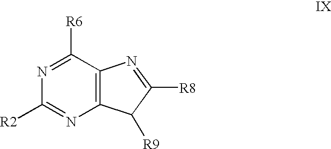

wherein R=H and R2, R6 and R9 are as defined above.

In another preferred embodiment the invention provides purine derivatives as represented by formula IX wherein R6=H and R2, R8 and R9 are as defined above.

In another preferred embodiment the invention provides purine derivatives as represented by formula IX wherein R2=H and R6, R8 and R9 are as defined above.

In another preferred embodiment the invention provides purine derivatives as represented by formula X

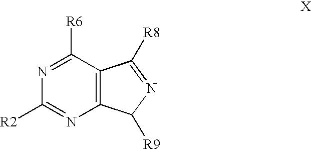

wherein R8=H and R2, R6 and R9 are as defined above.

In another preferred embodiment the invention provides purine derivatives as represented by formula X wherein R6=H and R2, R8 and R9 are as defined above.

In another preferred embodiment the invention provides purine derivatives as represented by formula X wherein R2=H and R6, R8 and R9 are as defined above.

In another preferred embodiment the invention provides purine derivatives as represented by formula XV

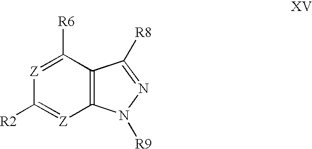

wherein R8=H and R2, R6 and R9 are as defined above.

In another preferred embodiment the invention provides purine derivatives as represented by formula XV wherein R6=H and R2, R8 and R9 are as defined above.

In another preferred embodiment the invention provides purine derivatives as represented by formula XV wherein R2=H and R6, R8 and R9 are as defined above.

In another preferred embodiment the invention provides purine derivatives as represented by formula XVI

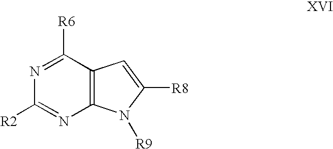

wherein R8=H and R2, R6 and R9 are as defined above.

In another preferred embodiment the invention provides purine derivatives as represented by formula XVI wherein R6=H and R2, R8 and R9 are as defined above.

In another preferred embodiment the invention provides purine derivatives as represented by formula XVI wherein R2=H and R6, R8 and R9 are as defined above.

In another preferred embodiment the invention provides purine derivatives as represented by formula XVII

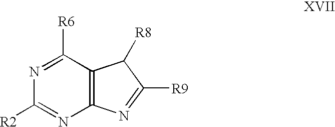

wherein R8=H and R2, R6 and R9 are as defined above.

In another preferred embodiment the invention provides purine derivatives as represented by formula XVII wherein R6=H and R2, R8 and R9 are as defined above.

In another preferred embodiment the invention provides purine derivatives as represented by formula XVII wherein R2=H and R6, R8 and R9 are as defined above.

The following purine derivatives are particularly preferred:

6-(3,4-dihydroxybenzyl)aminopurine, 6-(3,4-dihydroxybenzyl)amino-9-isopropylpurine, 2-(1-hydroxymethylpropylamino)-6-(3,4-dihydroxybenzyl)amino-9-isopropyl-purine, 2-(R)-(2-hydroxymethylpyrrolidine-1-yl)-6-(3,4-dihydroxybenzyl)amino-9-ispopropylpurine, 2-(2-aminopropylamino)-6-(3,4-dihydroxybenzyl)amino-9-isopropyl-purine, 2-(2-hydroxypropylamino)-6-(3,4-dihydroxybenzyl)-amino-9-isopropylpurine, 2-(R)-(1-isopropyl-2-hydroxyethylamino)-6-(3,4-dihydroxybenzyl)amino-9-isopropyl-purine, 6-[N-(3,4-dihydroxybenzyl)-N-methyl]amino-9-isopropylpurine, 6-[N-(3,4-dihydroxybenzyl)-N-methyl]-aminopurine, 6-[N-(3,4-dihydroxybenzyl)-N-methyl]amino-8-fluoropurine, 6-[N-(3,4-dihydroxybenzyl)-N-methyl)amino-9-isopropylpurine 2-(2-hydroxypropylamino)-6-(N-(3,4-dihydroxybenzyl)-N-methyl]amino-9-isopropylpurine, 2-(R)-(1-isopropyl-2-hydroxyethylamino-6-[N-(3,4-dihydroxybenzyl)-N-methyl]amino-9-isopropylpurine, 6-[1-(3,4-dihydroxyphenyl)ethyl]amino-9-isopropylpurine, 6-[1-(3,4-dihydroxyphenyl)ethyl]aminopurine, 6-[1-(3,4-dihydroxyphenyl)ethyl]amino-8-fluoropurine, 6-[1-(3,4-dihydroxyphenyl)ethyl]amino-9-isopropylpurine 2-(2-hydroxypropylamino)-6-[1-(3,4-dihydroxyphenyl) ethyl]amino-9-isopropylpurine, 2-(R)-(1-isopropyl-2-hydroxyethylamino-6-[1-(3,4-dihydroxyphenyl)ethyl]amino-9-isopropylpurine, 2-(R)-(1-isopropyl-2-hydroxyethylamino-6-[N-(2-(3,4-dihydroxyfenyl)ethyl)-N-methyl]amino-9-isopropylpurine, 6-[N-(2-(3,4-dihydroxyfenyl)ethyl)-N-methyl]amino-9-isopropylpurine, 6-[N-(2-(3,4-dihydroxyfenyl)ethyl)-N-methyl]-amino-8-bromo-9-isopropylpurine, 6-[N-(2-(3,4-dihydroxyfenyl)ethyl)-N-methyl]aminopurine, 6-(R,S)-hydroxy-1-phenylethylamino-9-isopropylpurine, 6-[(R,S)-(1-phenyl-2-hydroxyethyl)amino2-(1R-isopropyl-2-hydroxyethylamino)-6-[(R)-(1-phenyl-2-hydroxyethyl)amino]-9-isopropylpurine]purine, 2-(R)-(1-isopropyl-2-hydroxyethylamino-6-[(R,S)-(1-phenyl-2-hydroxyethyl)amino]-isopropylpurine, 2-chloro-6-[(R,S)-(1-phenyl-2-hydroxyethyl)amino]-9-isopropylpurine, 2-chloro-6-[(R,S)-(1-phenyl-2-hydroxyethyl)amino]purine.

The invention also relates to optical isomers and racemic mixtures, and, as the case may be, geometric isomers of the above-defined derivatives, in particular the (R) or (S) isomers of 2-(R)-(1-isopropyl-2-hydroxyethylamino)-6-[1-(3,4-dihydroxyphenyl)ethyl]amino-9-isopropylpurine, 2-(R)-(1-isopropyl-2-hydroxyethylamino)-6-(3,4-dihydroxybenzyl)amino-9-isopropylpurine, 2-(R)-[2-hydroxymethylpyrrolidine-1-yl]-6-(3,4-dihydroxybenzyl)-amino-9-isopropylpurine, 2-(1R-isopropyl-2-hydroxyethylamino)-6-[(S)-(1-phenyl-2-hydroxyethyl)amino]-9-isopropylpurine, 2-(1S-isopropyl-2-hydroxyethylamino)-6-[(S)-(1-phenyl-2-hydroxyethyl)amino]-9-isopropylpurine, 2-amino-6-[L-(3,4-dihydroxyphenyl)ethyl]amino-9-(R)-(2-phosphonomethoxypropyl)purine, 6-[N-(2-(3,4-dihydroxyfenyl)ethyl)-N-methyl]amino-9-(R)-(2-phosphonomethoxypropyl)purine, 2-amino-6-(3,4-dihydroxybenzyl)amino-9-(R)-(2-phosphonomethoxypropyl)purine, 6-[(R)-(1-phenyl-2-hydroxyethyl)amino]-8-chlor-9-(R)-(2-phosphonomethoxypropyl)purine.

The purine derivatives of the invention per se, or as intermediates in the preparation of novel compounds, have a wide variety of diagnostic, therapeutic and industrial utilities.

The compounds of this invention are for example suitable as intermediates for use in the preparation of affinity absorption matrices that harness the chemical properties of the compound's substituent groups. For example, the phosphonate groups in matrix-bound form are useful in the chromatographic separation of positively charged molecules. Other immobilised forms of the purine derivatives of the invention are for example useful in purifying proteins, e.g., cell cycle enzymes (such as cdks), or enzymes involved in the recognition of the compounds of this invention, e.g. transport proteins. Suitable methods of incorporation of the compounds of this invention into polymeric resins will be readily apparent to the skilled artisan. The compounds are for instance incorporated by cross-linking the hydroxyl groups of the phosphonate or hydroxymethyl substituents using cross-linking agents heretofore known. Linking through a group other than the heterocyclic base will produce a resin that may for example be useful in hydrophobic affinity chromatography.

The purine derivatives according to the invention can also be used as a modulator of α- and β-adrenergic and purinergic receptors. In a preferred embodiment, this invention thus provides a method for inhibiting or stimulating the signal transduction of adrenergic and purinergic receptors in mammals comprising administering a therapeutically effective amount of the composition of claim 1 to the mammal. The inhibiting and stimulating molecules are for example useful for treating inflammatory diseases and asthma, cardiovascular neurodegenerative and inflammatory diseases.

In another embodiment, this invention provides purine derivatives useful for treating fungal infections in humans, animal, and in plants.

The 2-, 6-, 8-, 9-monosubstituted, 2,6-, 2,9-, 6,8-, 6,9-disubstituted and 2,6,8-2,6,9-, 6,8,9-trisubstituted purine derivatives of the invention having at least one amine substituted with a catechol or related group (1,2-dihydroxybenzene) and related aza-deaza analogues result in the acquisition of extremely high potency against viruses, in particular DNA viruses. Moreover, surprisingly the chirally enriched or pure (S)-enantiomer is antivirally active. Heretofore, only the (R)-enantiomer was notably antivirally active, and then only against the retroviruses.

In another preferred embodiment of the invention, a method is provided for inhibiting cdks, and/or or cell proliferation and/or for inducing apoptosis in mammals, comprising administering a therapeutically effective amount of the compound according to the invention to the mammal. The cdk inhibiting molecules are useful for treating disorders, some of them involving cell proliferation, such as cancer, restenosis, rheumatoid arthritis, lupus, type I diabetes, multiple sclerosis, Alzheimer's disease, growth of parasites (animal, protists), graft rejection (host versus graft disease), graft versus host disease, and gout. The purine derivatives of the formula I and II and their pharmaceutically acceptable salts for example selectively inhibit cdks, in particular the enzyme p34^{cdc2}/cyclin B kinase and related cdks (cdk2, cdk5, cdk7, cdk9, erk1, erk2). In addition to other cdc2-related kinases, this kinase controls certain steps of cell division cycles, in particular the transition from G₁ phase into the S phase and in particular the transition from the G₂ phase into the M-phase. The compounds of formula I and II, and their pharmaceutically acceptable salts advantageously are used as antimitotic compounds, for example for the treatment of proliferative diseases, such as cancer and restenosis. In very low concentrations (micromolar and lower), they are capable of inhibiting cell cycle transitions (G₁/S, G₂/M, M-phase/metaphase) in different animal bodies and embryos.

Furthermore, the compounds are useful in treating auto-immune diseases, e.g. rheumatoid arthritis, lupus, type I diabetes, multiple sclerosis, etc.; in treating Alzheimer's disease, cardiovascular disease such as restenosis, graft rejection (host vs. graft disease), graft vs. host disease, gout, and in treating polycystic kidney disease, cancer and other proliferative diseases of which the pathogenesis involves abnormal cell proliferation.

The purine derivatives according to the invention also are potent and specific inhibitors of IκB-α kinase which prevents signal induced NF-κB activation and cytokine synthesis in vitro and in vivo. Such inhibitors inhibit synthesis of cytokines and adhesion proteins the synthesis of which is transcriptionally regulated by NF-κB. Pro-inflammatory cytokines such as IL-1, IL-6, TNF and adhesion proteins (e.g. ICAM, VCAM and selections) belong to this class of molecules and have been implicated in the pathogenesis of inflammatory diseases. Thus a potent inhibitor of IκB-α kinase is useful in the clinical management of diseases where NF-κB activation is required for disease induction. The compounds of the invention also affect the activation and/or signal transduction of α- and β-adrenergicreceptors, e.g. phosphatidyl turnover and cyclic AMP synthesis respectively. Activation of β-adrenergic receptors has an anti-inflammatory effect by decreasing the cytokine production of macrophages, astrocytes, and by preventing an increase in vascular permeability. On the other hand, a decreased β-adrenergic receptor activation is useful in diseases like multiple sclerosis and rheumatoid arthritis. The novel compounds also affect P2-purinergic receptor activation linked to phosphatidyl turnover and inhibition of activation of cyclic AMP synthesis or P1-purinergic receptor activation positively or negatively coupled to the activation of adenylate cyclase depending on the receptor subtype.

Modulation of purinergic receptor signalling may be useful in cerebral ischaemia, stroke, treatments of neurodegenerative diseases (e.g. Parkinson's disease), renal failure, treatment of lung dysfunction, and in inhibition of cancer growth.

The invention further provides novel compounds activating p53, the mammal cell's own natural brake gene for stopping uncontrolled cell proliferation (cancer), thus being able to switch off the cancer. p53 as well as retinoblastoma (Rb) are two well characterised tumour suppressors whose inactivation may lead to uncontrolled cell proliferation and malignancy. Phosphorylation of these two proteins, which are involved in cell cycle regulatory mechanisms, is known to modulate their function. A potent cdk-inhibitor thus represents a good tool for treatment of cancers due to induction of wild type p53 protein in cancers expressing mutant p53.

Studies carried out on the derivatives of the invention have demonstrated, in addition, the strong effect of the purine derivativs on apoptosis of many cancer cell lines. It has been demonstrated that apoptosis can be induced at stage G₁ or G₂ and following damage of the DNA, some cells stop at stage G₁ and a p53-dependent apoptotic pathway is then induced. In other situations, cells stop at G₂/M stage in response to damage caused to the DNA, and activation of a p53-independent apoptotic path is observed. This path has proved to be particularly significant in the therapy of tumours in which less active p53 is observed. By application of the purine derivatives of the invention, p53-independent apoptosis will be stimulated in cells which have stopped at stage G₂ through damage to the DNA using agents such as mitoxantrone or cis-platinum. The cdk inhibitors of this invention can thus increase the therapeutic potential of anti-tumour agents currently used.

The compounds of this invention can furthermore be terminally incorporated into oligonucleotides. If they contain a nonphosphonyl free hydroxyl group, they optionally are incorporated internally into the sequence of the oligonucleotide. Terminally incorporated diphosphonyl compounds of this invention which contain no free hydroxyl capable of participating in chain elongation also are useful in DNA sequencing in essentially the same manner as deoxy-NTPs have been used in the past (see example 8 of U.S. Pat. No. 5,276,143). The nucleotide analogues of the invention (when diphosphorylated) are useful as chain terminators for dideoxynucleotide-type DNA sequencing protocols, provided that the nucleotide analogue lacks a free hydroxyl group suitable for polymerase mediated chain elongation. These compounds will not have R=hydroxymethyl and do not posses a cyclic structure incorporating the phosphorus atom (although compounds having such excluded structures can be intermediates). The nucleotide analogue may be included in a kit with other reagents (such as Klenow polymerase or T4 polymerase, dNTPs, etc) needed for DNA sequencing (Otvos et al., “Nucl. Acids Res.” 1987: 15: 1763-1777

If the oligonucleotide-incorporated compound of this invention is binding-competent for its complementary sequence, i.e. if it is capable of base pairing, this nucleotide monomer will participate in hybridisation. It is not necessary, however, that the incorporated nucleotide analogue of this invention participates in hybridisation. If it is located at the terminus of the oligonucleotide, it will be useful as an immunological recognition site, or haptenic recognition site, to facilitate detection of the oligonucleotide by an antibody capable of binding the compound of this invention.

The compounds of this invention also are useful as linkers or spacers in preparation of affinity absorption matrices (as opposed to functioning as affinity moieties per se, as noted above), immobilised enzymes for process control, or immunoassay reagents. The compounds herein contain a multiplicity of functional groups that are suitable as sites for cross-linking desired substances. For example, it is conventional to link affinity reagents such as hormones, peptides, antibodies, drugs, and the like to insoluble substrates.

These insolubilised bound reagents are employed in known fashion to absorb binding partners for the affinity reagents from manufactured preparations, diagnostic samples and other impure mixtures. Similarly, immobilised enzymes are used to perform catalytic conversions with easy recovery of enzyme. Bifunctional compounds are commonly used to link analytes to detectable groups in preparing diagnostic reagents.

Many functional groups present in the compounds of this invention are suitable for use in cross-linking. For example, the phosphonic acid is used to form esters with alcohols or amides with amines. The R groups substituted with OH, azido (which is reduced to amino if desired before cross-linking) or vinyl are exemplary suitable sites. Similarly, the amino, halo, acyl and other reactive sites found on group R2, R6, and R9 are suitable. Suitable protection of reactive groups will be used where necessary while assembling the cross-linked reagent. In general, the compounds are used by linking through phosphonic acid or amino group to the hydroxyl or amino groups of the linking partner in the same fashion as shown, and covalently binding to the other binding partner through an R group. For example, a first binding partner such as a steroid hormone is esterified and then this conjugate is cross-linked through hydroxymethyl R to cyanogen bromide activated Sepharose, whereby an immobilised steroid is obtained. Other chemistries for conjugation are well known. See for example Maggio, “Enzyme-Immunoassay” (CRC, 1988, pp 71-135) and references cited therein.

The oligonucleotides of this invention may for example be labelled with any conventional detectable label, e.g. a fluorescent moiety such a fluorescein, radioisotopes such as ¹⁴C or ³H, stable free radicals, avidin, biotin and the like, all of which previously have been used as labels for immunoassays or diagnostic probes. The label may be present on the oligonucleotide or on the residue of an analogue of this invention. Suitable labelling methods are well known and are easily used with reactive groups such as hydroxyl, allyl and the like. A simple method is to label the compound of this invention with ³H by proton exchange. The compounds also may be biotinylated using conventional methods. See for instance U.S. Pat. No. 5,276,143 for analogous structures. The compounds of this invention, however, also are useful directly in diagnostic probe assays without an exogenous detectable label. In one embodiment of this alternative, antibodies are raised against the compounds of the invention. Such antibodies (which in turn are labelled or used in a double antibody configuration) bind to the analogue of this invention and thereby are useful in detecting its presence as label for a protein or oligonucleotide.

The compounds of the invention are useful for treatment of microbial infections, for treatment of tumours or for other indications described below. Microbial infections treatable by the compounds of this invention include viruses, parasites, yeast and fungi, but it is believed that the compounds are most effective against viruses, which constitutes the preferred utility. Exemplary viral infections include infections caused by DNA or RNA viruses including herpesviruses (herpes simplex virus type 1 (HSV-1), HSV-2, varicella zoster virus (VZV), Epstein-Barr virus (EBV), cytomegalovirus (CMV), human herpesvirus type 6 (HHV-6), HHV-7, HHV-8, bovine herpesvirus type 1, equine herpesvirus type 1, papillomaviruses (HPV types 1-55, including carcinogenic HPV), flaviviruses (including yellow fever virus, African swine fever virus and Japanese encephalitis virus), togaviruses (including Venezuelan equine encephalomyelitis virus), influenza viruses (types A-C), retroviruses (HIV-1, HIV-2, HTLV-I, HTLV-II, SIV, FeLV, FIV, MoMSV), adenoviruses (types 1-8), poxyiruses (vaccinia virus), enteroviruses (poliovirus types 1-3, Coxsackie, hepatitis A virus, and ECHO virus), gastroenteritis viruses (Norwalk viruses, rotaviruses), hantaviruses (Hantaan virus), polyomavirus, papovaviruses, rhinoviruses, parainfluenza virus types 1-4, rabies virus, respiratory synctial virus (RSV), hepatitis viruses A, B, C and E, and the like.

Antiviral purine derivatives of the invention preferably contain the following substituents at N9:

R9 is —(CH₂)ₙ—R9′, wherein n=1-2 and R9′ is —X(CH₂)ₘY, wherein

X is —O—, —S—, —NH— or —N(alkyl)-;

m is 1-2

Y is carboxyl, amido, sulfo, sulfamido, hydroxy, alkoxy, mercapto, alkylmercapto, amino, alkylamino, carbamino, —PO(OH)₂, —PO(alkyl)₂, —PO(Oalkyl)(NHalkyl), —PO(OH)(Oalkyl), —PO(OH)(NHalkyl); or

R9 is —(CH₂CHD)-R9′, wherein

X is —O—, —S—, —NH— or —N(alkyl)-;

m is 1-2

Y is carboxyl, amido, sulfo, sulfamido, hydroxy, alkoxy, mercapto, alkylmercapto, amino, alkylamino, carbamino, —PO(OH)₂, —PO(alkyl)₂, —PO(Oalkyl)(NHalkyl), —PO(OH)(Oalkyl), —PO(OH)(NHalkyl); and

D is alkyl, substituted alkyl, —PO(OH) 2, —PO(OH)(Oalkyl), —PO(OH)(NHalkyl).

The antiviral activity of the individual compounds may be determined by routine assay of antiviral (or other antimicrobial) activity using enzyme inhibition assays, tissue culture assays, animal model assays and the like, as will be understood by those skilled in the art.

Protozoan parasite infections to be treated using the compounds of the invention include infections caused by for example members of the subphyla Sarcomastigophora and Sporozoa of the phylum Protozoa. More particularly, the term protozoa as used herein include genera of parasitic protozoa, which are important to man, because they either cause disease in man or in his domestic animals. These genera for the most part are classified in the superclass Mastigophora of the subphylum Sarcomastiaophora and the class Telesporea of the subphylum Sporozoa in the classification according to Baker (1969). Illustrative genera of these parasitic protozoa include Histomonas, Pneumocystis, Trypanosoma, Giardia, Trichomonas, Eimeria, Isopora, Leishmania, Entamoeba, Toxoplasma and Plasmodium. Parasitic protozoans include Plasmodium falciparum, Plasmodium berghei, Plasmodium malariae, Plasmodium vivax, Leishmania braziliensis, Leishmania donovani, Trypanosoma cruzi, Trypanosoma brucei, Trypanosoma rhodesiense, Pneumocystis carinii, Entamoeba histolytica, Trichomonas vaginalis and the like (de Vries, E. et al., “Mol. Biochem. Parasitol.” 1991: 47:43-50) and trypanosomes (Kaminsky et al. “J. Parasitol.” 1994;80(6):1026-1030). The compounds in which R2, R6, R8 or R9 is CH₂OH and the purine ring is replaced by 3-deazaadenine are particularly interesting in the treatment of malarial parasites.

Compounds of the invention are also used to treat yeast or fungal infections caused by Candida clabrata, Candida tropicalis, Candida albicans, and other Candida species, Cryptococcus species including Cryptococcus neoformans, Blastomyces species including Blastomyces dermatitidis, Torulopsis species including Torulopsis glabrata, Coccidioides species including Coccidioides immitis, Aspergillus species and the like.

The compounds of the invention can furthermore be (1) applied to tissue culture systems to eliminate or reduce viral spread or growth during the production of biopharmaceutical or other products (such as proteins or vaccines), (2) used to eliminate or reduce viral spread or growth in clinical samples (such as blood), and (3) used to stop growth of tissue culture cells while leaving the cells to carry on with protein production.

The compounds of the invention furthermore have been found to suppress immunostimulation. Accordingly, they can suppress metabolic activities of T-lymphocytes stimulated by diverse agents, e.g. concanavalin A. The purine derivatives of the invention will find application in the treatment of for example autoimmune diseases, e.g. arthritis, or in suppression of transplant rejection.

In another preferred embodiment the invention provides a pharmaceutical composition one or more of the purine derivatives of the invention in an admixture with one or more pharmaceutical excipients or diluents.

### Processes for Preparation

The invention further provides a method to prepare the novel purine derivatives according to the invention.

The starting materials for the purine derivatives of formula I are 6-chloropurine and 2,6,-dichloropurine, prepared from hypoxanthine and hypoxanthine-1-N-oxide by chlorination with POCl₃. (Davoll and Blowy, J. Am. Chem. Soc. 1957, 73:2936). This starting material is also available from commercial sources (Sigma, Aldrich, Fluka, etc.). The compounds of the formula I may also be prepared from 2,6,8- and 6,8-dichloropurine prepared from uric acid by chlorination with POCl₃ (J. Am. Chem. Soc. 1958, 80:6671; J. Org. Chem. 1961, 26:447).

In one approach, the 6-substituted purines of formula I, wherein R6 substituents are as defined above, are prepared by reaction of 6-chloropurine with an appropriate amine, such as phenylglycinol, 2-, 3-, 4-hydroxybenzylamine, dihydroxybenzylamine, 4-amino-resorcinol, or 2-, 3-, 4-hydroxyaniline. 6-Chloropurine is dissolved in n-butanol and the appropriate R⁶-amine (1.5-5 eq.) and several-fold excess of triethylamine is used. After heating for several hours, the reaction mixture is cooled and the 6-substituted purine is obtained.

In another approach the 6,9-disubstituted purines of the formula I, wherein R6 and R9 substituents are as defined above, are prepared from 6-substituted is purines (in DMSO, or DMF) to which powdered calcium carbonate (aproximately 3 eq.) is added, followed by R⁹-halogen. After several hours or days of vigorous stirring the product is isolated by means of liquid chromatography.

In yet another approach the 6, 8, 9-trisubstituted purine derivatives of the formula I, wherein R6, R8 and R9 substituents are as defined above, are prepared from 6,9-disubstituted purines by S_{E} bromination (Br₂, CHCl₃, −20° C.) and subsequent S_{N} displacement of C⁸—Br by nucleophiles (5-30 equivalents of substituted amines, mercaptoderivatives with 2 equivalents of N-methylpyrrolidinone or N-ethyl-diisopropylamine, alcoholates) in DMAA at 100-180° C. An alternative approach is based on use of 6,8-dichloropurine as the starting compound. Nucleophilic substitution of C⁶—Cl (phenylglycinol, 2-, 3-, 4-hydroxybenzylamine, dihydroxybenzylamine, 4-amino-resorcinol, 2-, 3-,4-hydroxyaniline, mercaptoderivative, N-ethyl-diisopropylamine adition is used, alcoholate) is routinely achieved by reaction at 90-120° C. in n-butanol. After cooling the 8-chloro-6-substituted purines are obtained. Reactions with appropriate strong nucleophile; (10-30 equivalents of mercapto derivate, alcoholate, or substituted amine) in DMA or N-methylpyrrolidinone (150-200° C.) yielded desirable 6,8-disubstituted purines. Alkylation of these derivatives (R⁹-halogen; DMSO, or DMF; K₂Co₃ or NaH; 25° C.) is an alternative approach for preparation of 6,8,9-trisubstituted purines.

In yet another approach the 2,6-disubstituted purine derivatives of formula I, wherein R2 and R6 substituents are as defined above, are prepared from 2,6-dichloropurine by reacting 2,6-dichloropurine with appropriate nucleophile (phenylglycinol, 2-, 3-, 4-hydroxybenzylamine, dihydroxybenzylamine, 4-amino-resorcinol, 2-, 3-, 4-hydroxyaniline, substituted amines, mercaptoderivatives with 2 equivalents of N-methylpyrrolidone or N-ethyl-diisopropylamine, alcoholates) according to the method as described above for 6-chloropurine. Substitution of C²—Cl is then achieved by reaction with second nucleophile (5-30 equivalents of substituted amines, aminoalkanols; mercapto derivatives in the presence of N-methylpyrrolidinone or N-ethyl-diisopropylamine) at a temperature 160-180° C. The product is isolated by liquid chromatography or crystallized from n-BuOH or water.

In yet another approach, the 2,6,9-trisubstituted purine derivatives of formula I, wherein R2, R6 and R9 substituents are as defined above, are prepared by alkylation (K₂Co₃ DMSO, R⁹-halogen) of 2-chloro-6-substituted purines and subsequent reaction with R²-SH or R²-NH as described above for preparation of 2-chloro-6-substituted purine derivatives.

In another approach the 2,9-disubstituted purine derivatives of formula I, wherein R2 and R9 substituents are as defined above for a compound of formula I, are prepared from 2,6-dichloropurine in DMSO (or DMF) to which powdered potassium carbonate (5 equivalents) is added followed by R⁹-halogen (appr. 4 eq.). After one or two days of vigorous stirring, 9-alkylated-2,6-dichloropurine (detection on the basis of principal spot on TLC) is isolated by means of liquid chromatography. Selective hydrogenolysis of C⁶—Cl provides 2-chloro-9-alkylpurine (10 k Pd/BaSO₄₁ MeOH, Et₃N, 25° C., 2 h). The last nucleophilic substitution of C²—Cl is achieved by means of reaction in excess (2-30 eqv) of the desired nucleophile (substituted amines, mercapto derivatives with 2 equivalents of N-methylpyrrolidinone or N-ethyl-diisopropylamine, alcoholates) at a temperature 140-180° C. (2-48 h). 2,9-Disubstituted purines are then isolated by means of liquid chromatography.

In yet another approach, the substituted derivatives of formula I, wherein R2, R6 and R8 substituents are as defined above for a compound of formula I, are obtained by bromination of 2,6-disubstituted purines to get 2,6,8-trisubstituted purines (CHCl₃—MeOH, Br₂ −20° C.). Substitution of C⁸Br in 2,6-disubstituted-8-bromopurines is achieved by means of reaction with excess of nucleophile (5-30 equivalents of substituted amines, aminoalkanols; mercapto derivatives in the presence of N-methylpyrrolidinone or N-ethyl-diisopropylamine) at a temperature 160-180° C., DMAA may be used for solution. 2,6,8-trisubstituted purine derivatives are then isolated by means of liquid chromatography.

Similarly, bromination of trisubstituted derivatives of formula XI, wherein R2=Cl and R6, R9 are as defined above for a compound of formula I, gave the derivative in which R2=Cl and R8=Br. These substituents can be used in reaction either with the same nucleophile, or step by step with two various nucleophiles.

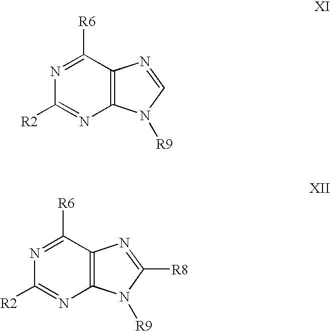

In yet another approach, the substituted purine derivatives of formula I, wherein R6=NH₂ or R2-NH₂ and R8, R9 substituents are as defined above for a compound of formula I, can be diazotated and transferred to halogen derivative with amylnitrite/CH₂Br₂ or with amylnitrite/CHI3. The halogen can be hydrogenolyzed with Pd catalyst/H₂ to prepare the R6, R8, R9- or R2, R6, R8-trisubstituted purine derivatives of formulas XIII or XIV.

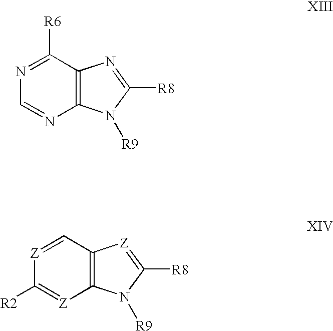

PMP and PME nucleotides are prepared by methods known for example from WO 94/03467, WO95/07920 and WO 96/33200. In general, the 6,8-chloropurine is first alkylated in DMF, either in the presence of an equivalent amount of sodium hydride or cesium carbonate at 60-100° C. The products are then isolated by chromatography on silica gel and crystallised from ethyl acetate by slow addition of petroleum ether until crystallisation occurs (the 2-amino-6-chloropurinyl PME/PMP compounds are crystalline, but the 6-chloropurinyl PME/PMP compounds are oils). The obtained 6-chloro compound is treated in ethanol solution with an excess (5 to 10 times) of the corresponding amine under reflux. The reaction is followed by TLC or HPLC analysis. The mixture is then evaporated, deionized on a cation exchanger column (Dowex 50), washed with 20% aqueous methanol, and the compound freed by the use of 2.5% ammonia in 20% aqueous methanol. The eluate is evaporated and dried over phosphorus pentoxide, and the residue is treated with 10% (v/v) bromotrimethylsilane in acetonitrile (5 ml per mM of compound) in order to deprotect the hydroxyl groups. The mixture is allowed to stand overnight and worked up in usual way. PMP/PME nucleotides can be easily brominated at R8 and subsequently modified at this position as described above for trisubstituted purines.

In an alternative method for preparing the compounds of the present invention, 2,6,8-trichloropurine is treated for 3-12 h with excess (5-10 fold) of primary or secondary amine in absolute ethanol or methanol at reflux temperature or in an autoclave at 100-120° C. The residue is purified by crystallisation, deionization on a cation exchange resin or by silica gel chromatography. The obtained 6-substituted purine derivative is pre-treated in dimethylformamide solution with one-half molar equivalent of cesium carbonate, one molar equivalent sodium hydride for 1 h at 100° C., and the appropriate phosphoro-organic synthon, used for example for the preparation of PME-, (R)-PMP or (S)-PMP derivatives (1.1-1.5 molar equivalents), is added to the mixture. The mixture is heated at 100-120° C. for 8-16 h, stripped off the solvent and the diester intermediate isolated by silica gel chromatography. The further treatment with bromotrimethylsilane and purification is performed as described above. It is not essential to employ the phosphonyl-protecting group where it is expected that the R6-substituent may be labile to the TMS deprotection. In this case, the free acid is used as the starting material for addition of the amine.

### Therapeutic Administration

Suitable routes for administration of the purine 35 derivatives according to the invention for use in the treatment of the human or animal body include for example oral, rectal, topical (including dermal, ocular, buccal and sublingual), vaginal and parenteral routes (including subcutaneous, intramuscular, intravitreous, intravenous, intradermal, intrathecal and epidural). The preferred route of administration will depend upon the condition of the patient, the toxicity of the specific derivative used and the site of infection, among other considerations known to the clinician.

In a preferred embodiment of the invention the pharmaceutical composition comprises about 1% to about 95% of one or more of the purine derivatives of the invention as the active ingredient and at least a pharmaceutically acceptable carrier or diluent, single-dose forms of administration preferably comprising about 20% to about 90% of the active ingredient and administration forms which are not single-dose preferably comprising about 5% to about 20% of the active ingredient. Unit dose forms are, for example, coated tablets, tablets, ampoules, vials, suppositories or capsules. Other forms of administration include, for example, ointments, creams, pastes, foams, tinctures, lipsticks, drops, sprays, dispersions and the like. In a preferred embodiment the purine derivatives are incorporated in capsules comprising about 0.05 g to about 1.0 g of the active ingredient.

The pharmaceutical compositions of the present invention are prepared in a manner known per se, for example by means of convectional mixing, granulating, coating, dissolving or lyophilising processes.

Preferably, solutions of the active ingredient, and in addition also suspensions or dispersions, in particular isotonic aqueous solutions, dispersions or suspensions, are used, it being possible for these to be prepared before use, as for example in the case of lyophilised compositions which comprise the active substance by itself or together with a carrier, for example mannitol. The pharmaceutical compositions can be sterilised and/or comprise excipients, for example preservatives, stabilisers, wetting agents and/or emulsifiers, solubilizing agents, salts for regulating the osmotic pressure and/or buffers, and they can be prepared in a manner known per se, for example by means of convectional dissolving or lyophilising processes. The solutions or suspensions can comprise viscosity-increasing substances, such as sodium carboxymethylcellulose, carboxymethylcellulose, dextran, polyvinylpyrrolidone or gelatin.

Suspensions-in-oil comprise, as the oily component, the vegetable, synthetic or semi-synthetic oils customary for injection purposes. Oils to be used in the invention preferably include liquid fatty acid esters which contain, as the acid component, a long-chain fatty acid having 8-22, in particular 12-22, carbon atoms, for example lauric acid, tridecylic acid, myristic acid, pentadecylic acid, palmitic acid, margaric acid, stearic acid, arachidonic acid, behenic acid or corresponding unsaturated acids, for example oleic acid, elaidic acid, euric acid, brasidic acid or linoleic acid, if appropriate with the addition of antioxidants, for example vitamin E, β-carotene or 3,5-di-tert-butyl-4-hydroxytoluene. The alcohol component of these fatty acid esters preferably has no more than 6 carbon atoms and is mono- or polyhydric, for example mono-, di- or trihydric alcohol, for example methanol, ethanol, propanol, butanol, or pentanol, or isomers thereof, but in particular glycol and glycerol. Suitable fatty acid esters are for example: ethyl oleate, isopropyl myristate, isopropyl palmitate, “Labrafil M 2375” (polyoxyethylene glycerol trioleate from Gattefossé, Paris), “Labrafil M 1944 CS” (unsaturated polyglycolated glycerides prepared by alcoholysis of apricot kernel oil and made of glycerides and polyethylene glycol esters; from Gattefossé, Paris), “Labrasol” (saturated polyglycolated glycerides prepared by an alcoholysis of TCM and made up of glycerides and polyethylene glycol esters; from Gattefossé, Paris) and/or “Miglyol 812” (triglyceride of saturated fatty acids of chain length C₈ to C₁₂ from Hüls AG, Germany), and in particular vegetable oils, such as cottonseed oil, almond oil, olive oil, castor oil, sesame oil, soybean oil and, in particular, groundnut oil.

The preparation of the injection compositions is carried out in customary manner under sterile conditions, as are bottling, for example in ampoules or vials, and closing of the containers.

Pharmaceutical compositions for oral use can for example be obtained by combining the active ingredient with one or more solid carriers, if appropriate granulating the resulting mixture, and, if desired, processing the mixture or granules to tablets or coated tablet cores, if appropriate by addition of additional excipients. Suitable carriers are, in particular, fillers, such as sugars, for example lactose, sucrose, mannitol or sorbitol, cellulose preparations and/or calcium phosphates, for example tricalcium diphosphate, or calcium hydrogen phosphate, and furthermore binders, such as starches, for example maize, wheat, rice or potato starch, methylcellulose, hydroxypropylmethylcellulose, sodium carboxymethylcellulose and/or polyvinylpyrrolidine, and/or, if desired, desintegrators, such as the above mentioned starches, and furthermore carboxymethyl-starch, cross-linked polyvinylpyrrolidone, alginic acid or a salt thereof, such as sodium alginate. Additional excipients are, in particular, flow regulators and lubricants, for example salicylic acid, talc, stearic acid or salts thereof, such as magnesium stearate or calcium stearate, and/or polyethylene glycol, or derivatives thereof.

Coated tablet cores can be provided with suitable coatings which, if appropriate, are resistant to gastric juice, the coatings used being, inter alia, concentrated sugar solutions, which, if appropriate, comprise gum arabic, talc, polyvinylpyrrolidine, polyethylene glycol and/or titanium dioxide, coating solutions in suitable organic solvents or solvent mixtures or, for the preparation of coatings which are resistant to gastric juice, solutions of suitable cellulose preparations, such as acetylcellulose phthalate or hydroxypropylmethylcellulose phthalate. Dyes or pigments can be admixed to the tablets or coated tablet coatings, for example for identification or characterisation of different doses of the active ingredient.

Pharmaceutical compositions, which can be used orally, are also hard capsules of gelatin and soft, closed capsules of gelatin and a plasticiser, such as glycerol or sorbitol. The hard capsules can contain the active ingredient in the form of granules, mixed for example with fillers, such as maize starch, binders and/or lubricants, such as talc or magnesium stearate, and stabilisers if appropriate. In soft capsules, the active ingredient is preferably dissolved or suspended in suitable liquid excipients, such as greasy oils, paraffin oil or liquid polyethylene glycols or fatty acid esters of ethylene glycol or propylene glycol, it being likewise possible to add stabilisers and detergents, for example of the polyethylene sorbitan fatty acid ester type.

Other oral forms of administration are, for example, syrups prepared in the customary manner, which comprise the active ingredient, for example, in suspended form and in a concentration of about 5% to 20%, preferably about 10% or in a similar concentration which results in a suitable individual dose, for example, when 5 or 10 ml are measured out. Other forms are, for example, also pulverulent or liquid concentrates for preparing shakes, for example in milk. Such concentrates can also be packed in unit dose quantities.

Pharmaceutical compositions, which can be used rectally, are, for example, suppositories that comprise a combination of the active ingredient with a suppository base. Suitable suppository bases are, for example, naturally occurring or synthetic triglycerides, paraffin hydrocarbons, polyethylene glycols or higher alkanols.

Compositions which are suitable for parenteral administration are aqueous solutions of an active ingredient in water-soluble form, for example of water-soluble salt, or aqueous injection suspensions, which may comprise viscosity-increasing substances, for example sodium carboxymethylcellulose, sorbitol and/or dextran, and if appropriate stabilisers. The active ingredient can also be present in the form of a lyophilisate, if appropriate together with excipients, and be dissolved prior to parenteral administration by addition of suitable solvents. Solutions such as are used, for example, for parenteral administration can also be used as infusion solutions. Preferred preservatives are, for example antioxidants, such as ascorbic acid, or microbicides, such as sorbic or benzoic acid.

Ointments are oil-in-water emulsions, which comprise not more than 70%, but preferably 20-50% of water or aqueous phase. The fatty phase may for example consist of hydrocarbons, for example vaseline, paraffin oil or hard paraffin's, which preferably comprise suitable hydroxy compounds, such as fatty alcohol's or esters thereof, for example cetyl alcohol or wool wax alcohols, such as wool wax, to improve the water-binding capacity. Emulsifiers are corresponding lipophilic substances, such as sorbitan fatty acid esters (Spans), for example sorbitan oleate and/or sorbitan isostearate. Additives to the aqueous phase are, for example, humectants, such as polyalcohols, for example glycerol, propylene glycol, sorbitol and/or polyethylene glycol, or preservatives and odoriferous substances.

Fatty ointments are anhydrous and may comprise, as the base, in particular, hydrocarbons, for example paraffin, vaseline or paraffin oil, and furthermore naturally occurring or semi-synthetic fats, for example hydrogenated coconut-fatty acid triglycerides, or, preferably, hydrogenated oils, for example hydrogenated groundnut or castor oil, and furthermore fatty acid partial esters of glycerol, for example glycerol mono- and/or distearate, and for example, the fatty alcohols. They also may contain emulsifiers and/or additives mentioned in connection with the ointments which increase uptake of water.

Creams are oil-in-water emulsions, which comprise more than 50% of water. Oily bases used are, in particular, fatty alcohols, for example lauryl, cetyl or stearyl alcohols, fatty acids, for example palmitic or stearic acid, liquid to solid waxes, for example isopropyl myristate, wool wax or beeswax, and/or hydrocarbons, for example vaseline (petrolatum) or paraffin oil. Emulsifiers are surface-active substances with predominantly hydrophilic properties, such as corresponding non-ionic emulsifiers, for example fatty acid esters of polyalcohols or ethyleneoxy adducts thereof, such as polyglyceric acid fatty acid esters or polyethylene sorbitan fatty esters (Tweens), and furthermore polyoxyethylene fatty alcohol ethers or polyoxyethylene fatty acid esters, or corresponding ionic emulsifiers, such as alkali metal salts of fatty alcohol sulfates, for example sodium lauryl sulfate, sodium cetyl sulfate or sodium stearyl sulfate, which are usually used in the presence of fatty alcohols, for example cetyl stearyl alcohol or stearyl alcohol. Additives to the aqueous phase are, inter alia, agents which prevent the creams from drying out, for example polyalcohols, such as glycerol, sorbitol, propylene glycol and/or polyethylene glycols, and furthermore preservatives and odoriferous substances.

Pastes are creams and ointments having secretion-absorbing powder constituents, such as metal oxides, for example titanium oxide or zinc oxide, and furthermore talc and/or aluminium silicates, which have the task of binding the moisture or secretions present.

Foams may be administered from pressurised containers and may be liquid oil-in-water emulsions resent in aerosol foam. As the propellant gases, alogenated hydrocarbons, such as polyhalogenated alkanes, for example dichlorofluoromethane and dichlorotetrafluoroethane, or, preferably, non-halogenated gaseous hydrocarbons, air, N₂O, or carbon dioxide are used. The oily phases and the additives used are, inter alia, those mentioned above for ointments and creams.

Tinctures and solutions usually comprise an aqueous-ethanolic base to which, for example, humectants for reducing evaporation, such as polyalcohols, for example glycerol, glycols and/or polyethylene glycol, and re-oiling substances, such as fatty acid esters with lower polyethylene glycols, i.e. lipophilic substances soluble in the aqueous mixture to substitute the fatty substances removed from the skin with the ethanol, and, if necessary, other excipients and additives, are admixed.

The pharmaceutical compositions according to the invention may also comprise veterinary compositions comprising at least one active ingredient as above defined together with a veterinary carrier therefor. Veterinary carriers are materials for administering the composition and may be solid, liquid or gaseous materials, which are inert or acceptable in the veterinary art and are compatible with the active ingredient. These veterinary compositions may be administered orally, parenterally or by any other desired route.

The invention also provides methods for treatment of several diseases, such as those disease states mentioned above. The compounds can be administered prophylactically or therapeutically as such or in the form of pharmaceutical compositions, preferably in an amount, which is effective against the diseases mentioned. With a warm-blooded animal, for example a human, requiring such treatment, the compounds are used, in particular, in the form of pharmaceutical composition.

The invention is further illustrated by the following Examples and figures, which only serve to illustrate the invention without limiting the scope thereof.

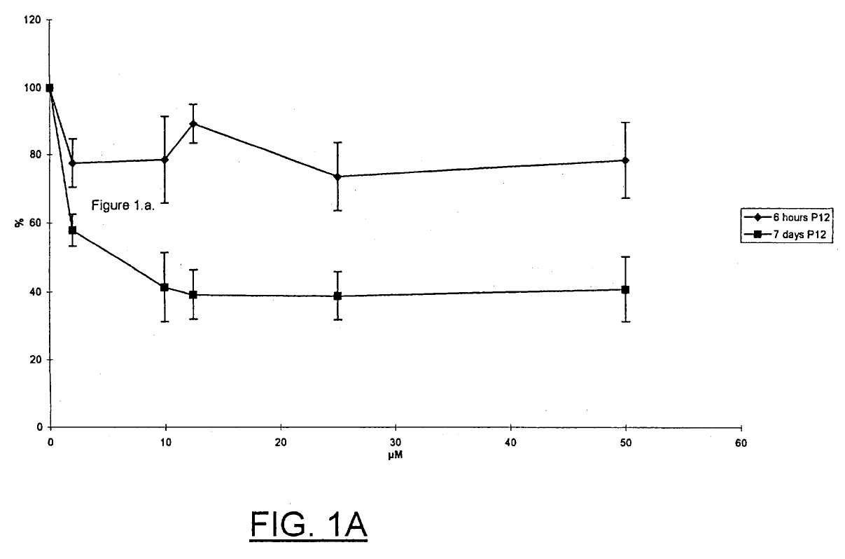

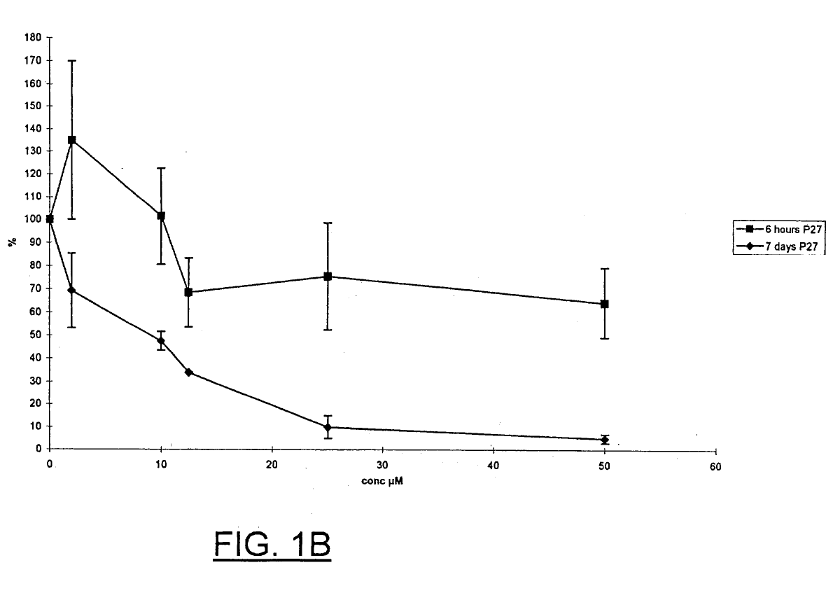

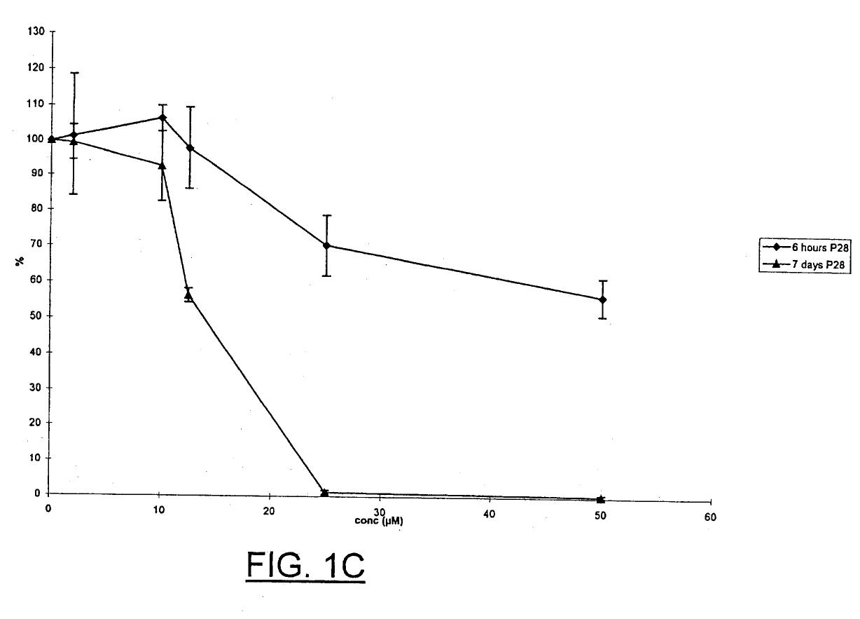

FIG. 1 shows dose-response curves of the purine derivatives of the invention. The response of the myeloid leukemia KG1 cell line to several compounds was demonstrated.

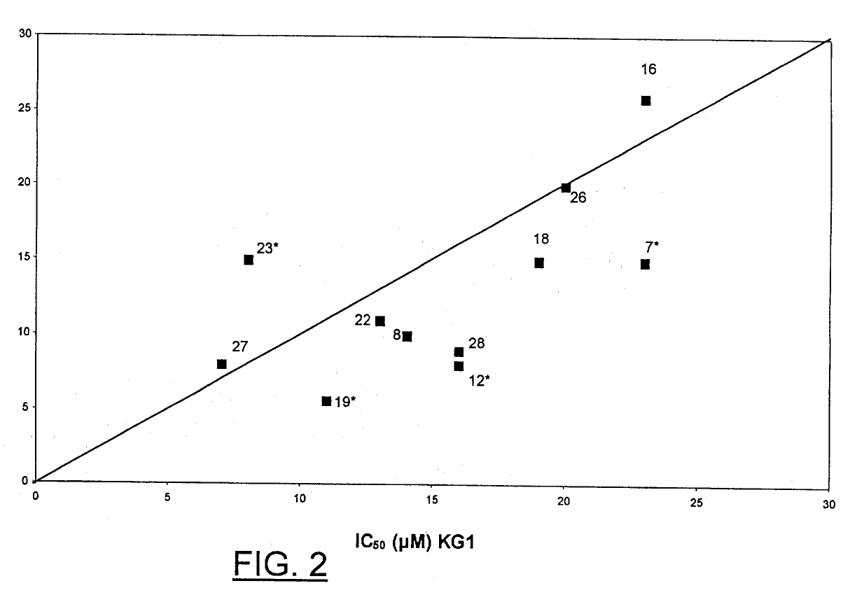

FIG. 2 shows a comparison between normal PHA-stimulated lymphocytes and hematopoietic cell lines, demonstrating that lymphocytes are more sensitive to some of the novel compounds than cell lines, with the exception of P23, that was significantly more effective on KG1 than on normal PBMC.

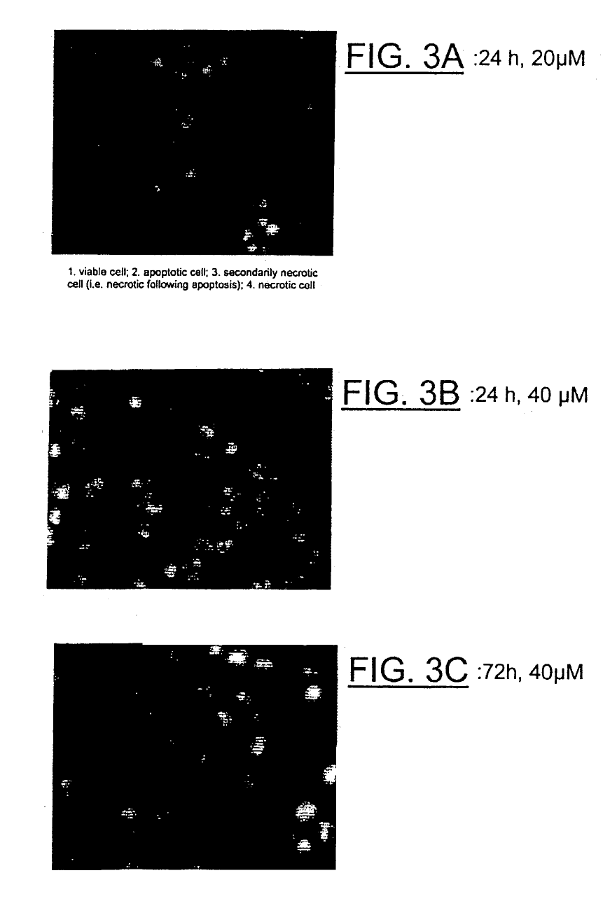

FIG. 3 shows microscopic results of induction of apoptosis in the Jurkat T-cell line incubated with compound P12 at different time points.

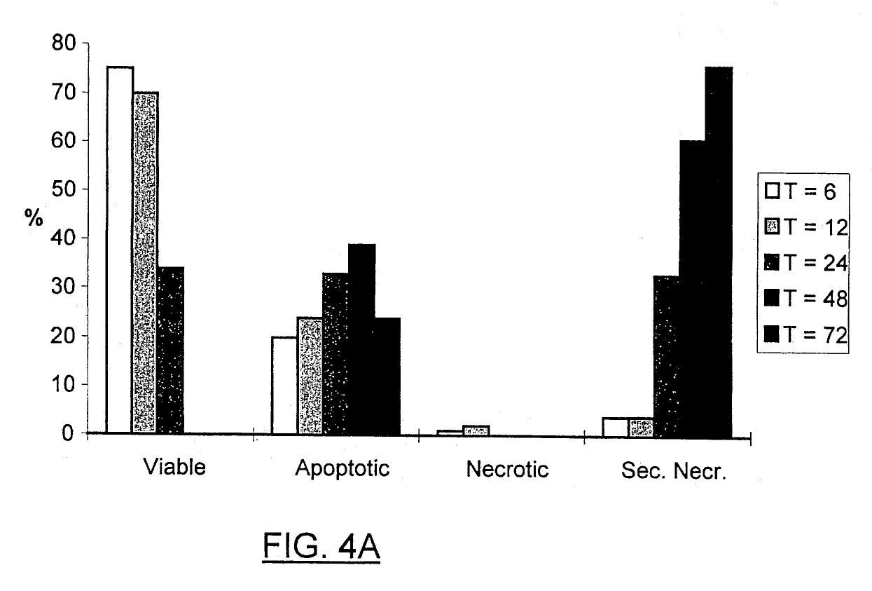

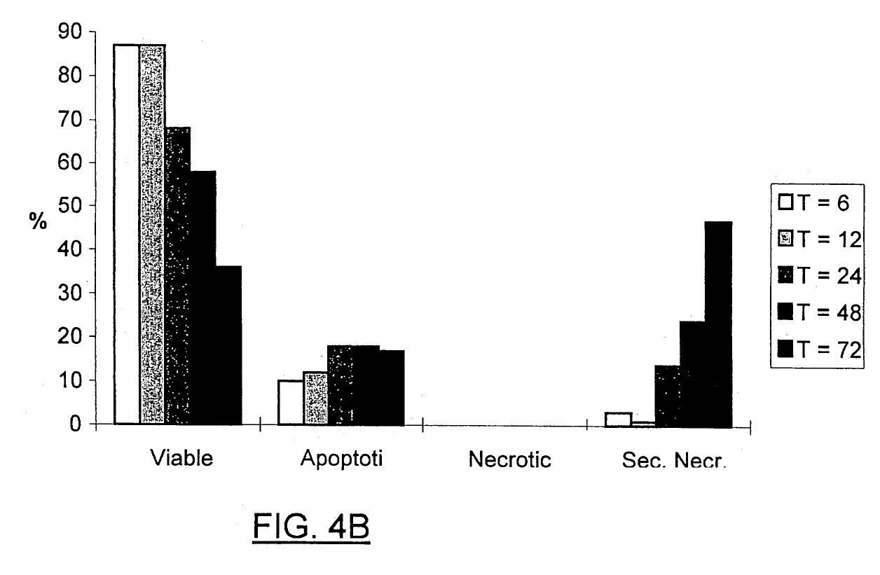

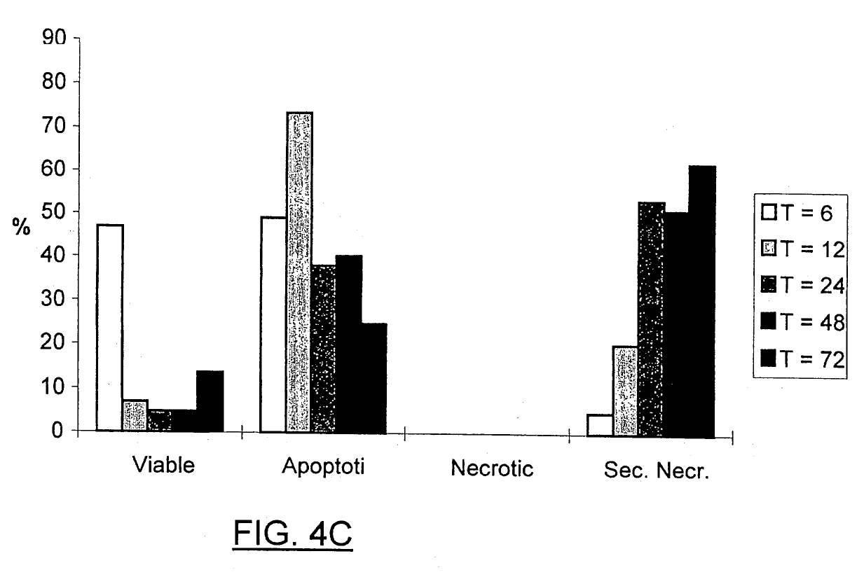

FIG. 4 shows the results of microscopic examination of KG1 cells incubated with P23, P27 and P28. Viable, apoptotic, necrotic, and secondarily necrotic cells were scored after 6, 12, 24, 48 and 72 hours of exposure to the compounds of the invention.

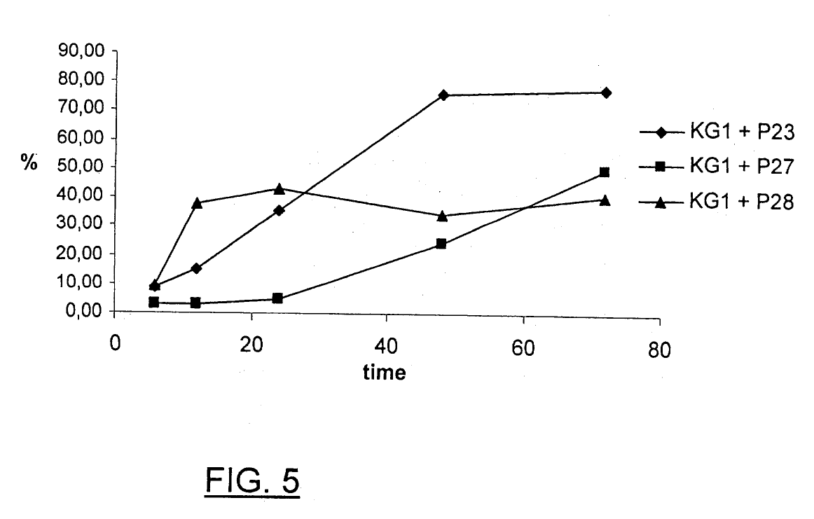

FIG. 5 is a graph showing the percentage of cells with DNA fragmentation (apoptosis) as detected by the TUNEL technique in cell line KG1 after incubation with P23, P27 and P28 over a period of 72 hours.

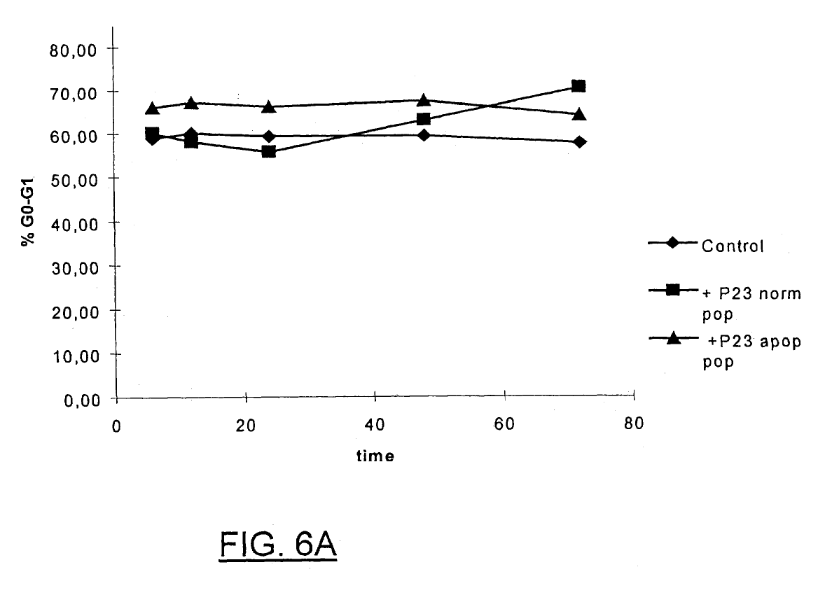

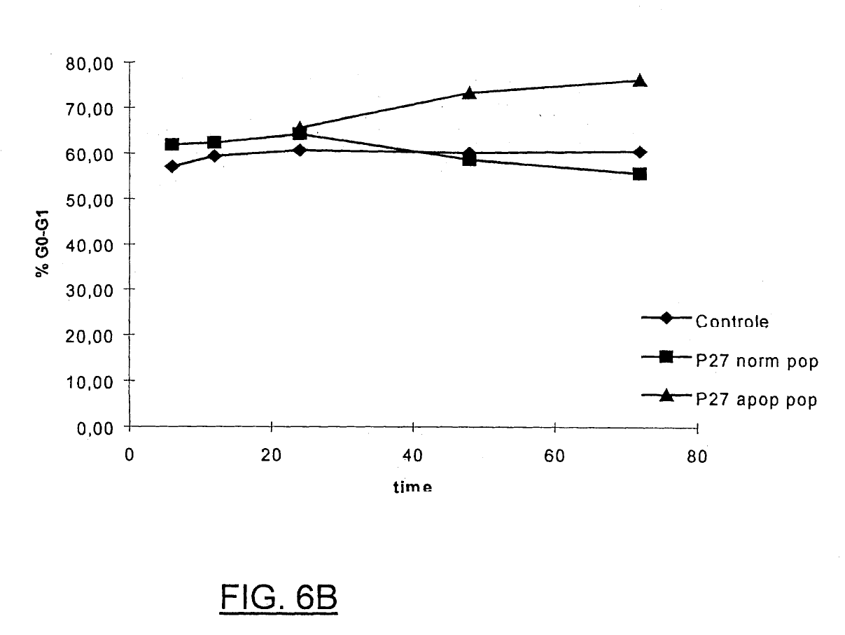

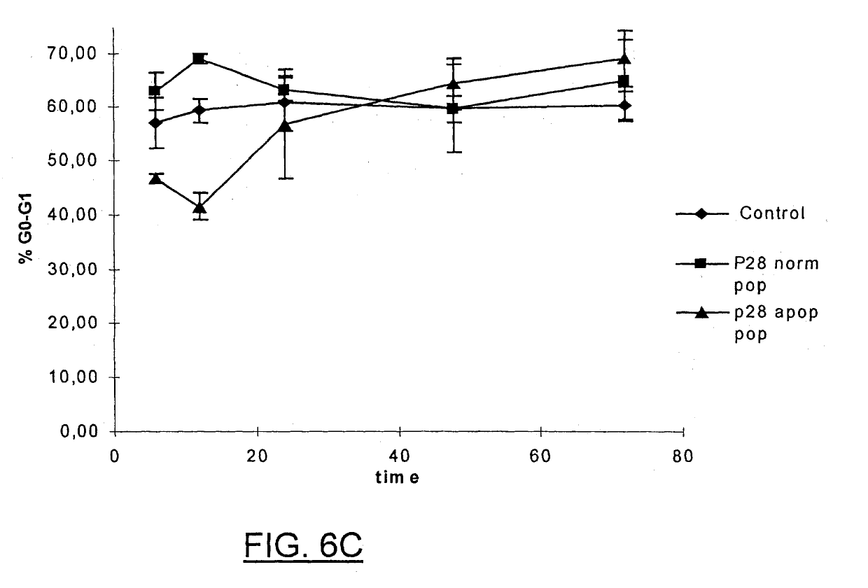

FIG. 6 shows graphs demonstrating the percentage of KG1 cells in G₀-G₁ phase as detected by DNA staining with ethidium bromide (EB); a) P23; b) P27; c) P28. TUNEL in combination with EB shows the percentage of apoptotic (apop pop) and non-apoptotic (norm pop) cells in G₀-G₁.

### EXAMPLES

### Example 1

6-[(RS)-(1-Phenyl-2-hydroxyethyl)amino]purine

6-Chloropurine (3 mmol, 0.47 g), (RS)-2-phenylglycinol (5 mmol, 0.7 g), and triethylamine (2 mL) were heated with stirring in 10 mL of 1-butanol (125° C., 3 h). After cooling, the volatile components were evaporated. The product was obtained by crystallization from acetic acid-ethyl acetate. Recrystallization from hot acetic acid gave the desired product; yield 82%, mp 130-138° C. MS(Waters/Micromas ZMD detector, direct inlet, MeOH+AcOH solution, ESI 20 eV): 256.4 (100%), [M+H]^{+.}. FTIR (Nicolet 205, KBr, cm⁻¹) 1695, 1623, 1607, 1587, 1411, 1368, 1317, 1291.

2-Chloro-6-[(S)-(1-phenyl-2-hydroxyethyl)amino]purine

2,6-Dichloropurine (1.66 mmol, 0.31 g), (S)-2-phenylglycinol (2 mmol, 0.27 g) and triethylamine (0.3 mL) were heated in 5 mL of 1-butanol (120° C., 1 h). After evaporation of the volatile components the product was isolated by means of column chromatography (silica gel, CHCl₃/MeOH/AcOH; 60/2.8/1.2). Crystallization from MeOH-Et₂O gave 0.33 g of the product; yield 86%; mp 205-210° C.; [α]_{D}=+21.9 (MeOH, c=0.22).

Other purine derivatives according to the invention which were prepared by the method of Example 1 are listed in table 1.

TABLE 1
Purine derivatives prepared by the method of
Example 1

| PURINE SUBSTITUENT | | |
|---|---|---|
| C2 | N6 | C8 |
| | [N-(3,4-dihydroxybenzyl-N-methyl]amino | |
| | 3,4-dihydroxybenzylamino | |
| | [(R,S)-(1-phenyl-2-hydroxyethyl)amino] | |
| Chloro | [N-(3,4-dihydroxybenzyl-N-methyl]amino | |
| Chloro | 3,4-dihydroxybenzylamino | |
| | [(R,S)-(1-phenyl-2-hydroxyethyl)amino] | |
| | [(R,S)-(1-phenyl-2-hydroxyethyl)amino] | Chloro |
| | [(R,S)-(1-phenyl-2-hydroxyethyl)amino] | |
| Chloro | [(R,S)-(1-phenyl-2-hydroxyethyl)amino] | Chloro |
| Chloro | [(R,S)-(1-phenyl-2-hydroxyethyl)amino] | |
| | [N-(3,4-dihydroxybenzyl-N-methyl]amino | Chloro |
| | [N-(3,4-dihydroxybenzyl-N-methyl]amino | Bromo |
| Chloro | [N-(3,4-dihydroxybenzyl-N-methyl]amino | Chloro |
| Chloro | [N-(3,4-dihydroxybenzyl-N-methyl]amino | Bromo |

### Example 2

2-chloro-9-isopropyl-6-[(S)-(1-phenyl-2-hydroxyethyl)amino]purine

2-chloro-6-[(S)-(1-phenyl-2-hydroxyethyl)amino]purine (0.6 mmol, 0.17 g), powdered potassium carbonate (1.4 mmol, 0.2 g) and isopropylbromide (4.2 mmol, 0.4 mL) were vigorously stirred in dry DMF (2 mL) for 24 hours. 2-bromopropane (0.4 mL) was added and the reaction was continued for another 48 hours. After evaporation of the volatile components the residue was partitioned between water and ethyl acetate. The organic layer was dried over sodium sulphate. The product was crystallized from diethylether (0.167 g). Yield 84%, mp 150-152° C., [α]_{D}=+19.2 (c=0.22; CHCl₃). MS EI (Jeol JMS-D100, 80 eV, 300 νA, 200° C., direct inlet): 331.1183 (Me^{+.}; C₁₅H₁₈N₅OCl, theor. 331.1200; 0.3), 301(48), 300.1013 (C₁₅H₁₅N₅Cl, theor. 300.1016; 89), 258(100),222(11), 195(6), 155(6), 153(9), 134(6), 119(18), 106(11), 91(9), 77(11).

2-(S)-(2-hydroxypropyl)amino-9-isopropyl-6-[(S)-(1-phenyl-2-hydroxyethyl)amino]purine

2-chloro-9-isopropyl-6-[(S)-(1-phenyl-2-hydroxyethyl)amino]purine (0.28 mmol, 94 mg) and (S)-2-hydroxypropylamine (2.8 mmol, 0.22 mL) were heated (sealed ampoule, 160° C., 3 h) in diglyme (0.5 mL). After evaporation of the solvent and an excess of amine, the product was purified by column chromatography (silica gel, 3% MeOH in CHCl₃). Crystallization from CHCl₃-Et₂O afforded 2-(S)-(2-hydroxyethyl)amino-9-isopropyl-6-[(RS)-(1-phenyl-2-hydroxyethyl)amino]purine (75 mg); yield 72%; mp 110-112° C.; [α]_{D}=+58.6 (c=0.2, CHCl₃). MS EI (Jeol JMS-D100, 80 eV, 300 νA, 200° C., direct inlet): 370.2129 (M^{+.}; C₁₉H₂₆N₆O, theor. 370.2117; 15), 352(8), 340(49), 339(100), 325(8), 321(18), 297(19), 296(23), 295(26), 282(11), 279(41), 205(40), 163(24), 134(17), 106(22), 91(15), 41(12). ¹H NMR (200 MHz, CDCl₃): 1.20 d (3H, J=6.4, CH₃CHOH), 1.52 d (6H, J=6.5, (CH₃)₂CH), 1.8 bs (1H, exch), 3.32 m (2H, CH₂NH), 4.00 d (2H, J=5.0, CH₂OH), 4.00 m (1H, CHOH), 4.59 sept (1H, J=6.5, CH(CH₃)₂), 5.16 t (1H, CHPh), 5.3 bs(1H, exch), 6.50 bs (1H, exch), 7.22-7.40 m (5H, ph), 7.50 s (1H, HC⁸)

2-(R)(2-hydroxypropyl)amino-9-isopropyl-6-[(S)-(1-phenyl-2-hydroxyethyl)amino]purine

This purine derivative was prepared in the same manner as the previous isomer with the exception that the (R)-2-hydroxypropylamine was used instead of (S)-2-hydroxypropylamine; yield 70%; mp 109-112° C.; [α]_{D}=+43.6 (c=0.15, CHCl₃). MS EI (Jeol JMS-D100, 80 eV, 300 νA, 200° C., direct inlet): 370.2129 (M^{+.}; C₁₉H₂₆N₆O, teor. 370.2117; 15), 352 (5), 340 (49), 339 (100), 325 (8), 321(18), 297(19), 296(23), 295(26), 282(11), 279(41), 205(40), 163(25), 134(17), 106(22), 91 (15), 41(12). ¹H NMR (200 MHz, CDCl₃): 1.20 d (3H, J=6.4, CH₃CHOH), 1.53 d (6H, J=6.5, (CH₃)₂CH), 1.8 bs (1H, exch), 3.32 m (2H, CH₂NH), 4.01 d (2H, J=5.0, CH₂OH), 4.00 m (1H, CHOH), 4.59 sept (1H, J=6.5, CH(CH₃)₂), 5.16 t (1H, CHPh), 5.3 bs (1H, exch), 6.50 bs (1H, exch), 7.22-7.40 m (5H, ph), 7.50 s (1H, HC3)

2-hexylamino-9-isopropyl-6-[(S)-(1-phenyl-2-hydroxyethyl)amino]purine

2-chloro-9-isopropyl-6-[(S)-(1-phenyl-2-hydroxyethyl)amino]purine (0.3 mmol, 100 mg) was heated in n-hexylamine (3 mL) (sealed ampoule, 160° C., 3 h). Excess of the amine was evaporated and the product was purified by column chromatography (silica gel, stepwise 0, 1, 2, % MeOH in CHCl₃). The syrup-like product (110 mg, 92) crystallized spontaneously after several weeks; mp was too low for measuring. MS EI (Jeol JMS-D100, 80 eV, 300 νA, 200° C., direct inlet): 396(M^{+.}; 12), 378(17), 377(10), 366(56), 365(100), 323(41), 307(10), 301(7), 295(10), 251(8), 239(14), 205(12), 163 (9), 134(16) 106(23). H NMR (400 MHz, CDCl₃): 0.907 t (3H, J=6.9, CH₃CH₂), 1.287-1.1420 m (4H, (CH₂)₂), 1.521-1.65 m (10H, (CH₂)₂+(CH₃)₂CH), 3.31-3.42 m (2H, CH₂N), 3.955-4.08 m (2H, CH₂OH), 4.635 sept (1H, J=6.8, CH(CH₃)₂), 4.74 bs (1H, NHCH₂), 5.315 bs (1H, CHCH₂OH), 7.28-7.42 m (5H, Ph), 7.496(1H, HC⁸). The 2D-COSY spectra were used for the structural assignment of proton signals.

Table 2 lists the purine derivatives according to the invention that were prepared by the method of Example 2.

TABLE 2
Purine derivatives prepared by the method of
Example 2
PURINE SUBSTITUENT

| C2 | N6 | N9 |
|---|---|---|
| | [(R,S)-(1-phenyl-2-hydroxyethyl)amino] | Isopropyl |
| Chloro | [(R,S)-(1-phenyl-2-hydroxyethyl)amino] | Isopropyl |
| 2-hydroxyethylamino | [(R,S)-(1-phenyl-2-hydroxyethyl)amino] | Isopropyl |
| 2-hydroxypropylamino | [(R,S)-(1-phenyl-2-hydroxyethyl)amino] | Isopropyl |
| 3-hydroxypropylamino | [(R,S)-(1-phenyl-2-hydroxyethyl)amino] | Isopropyl |
| (R)-1-(hydroxymethyl)- | [(R,S)-(1-phenyl-2-hydroxyethyl)amino] | Isopropyl |
| propylamino | | |
| [bis-(2-hydroxyethyl)]-amino | [(R,S)-(1-phenyl-2-hydroxyethyl)amino] | Isopropyl |
| 2-(1R-isopropyl-2- | [(R,S)-(1-phenyl-2-hydroxyethyl)amino] | Isopropyl |
| hydroxyethylamino) | | |
| | [N-(3,4-dihydroxybenzyl-N- | Isopropyl |
| | methyl]amino | |
| Chloro | [N-(3,4-dihydroxybenzyl-N- | Isopropyl |
| | methyl]amino | |
| 2-hydroxyethylamino | [N-(3,4-dihydroxybenzyl-N- | Isopropyl |
| | methyl]amino | |
| 3-hydroxypropylamino | [N-(3,4-dihydroxybenzyl-N- | Isopropyl |
| | methyl]amino | |
| (R)-1-(hydroxymethyl) | [N-(3,4-dihydroxybenzyl-N- | Isopropyl |
| propylamino | methyl]amino | |
| [bis-(2-hydroxyethyl)-amino | [N-(3,4-dihydroxybenzyl-N- | Isopropyl |
| | methyl]amino | |
| 2-(1R-isopropyl-2- | [N-(3,4-dihydroxybenzyl-N- | Isopropyl |
| hydroxyethylamino) | methyl]amino | |
| | 3,4-dihydroxybenzylamino | Isopropyl |
| Chloro | 3,4-dihydroxybenzylamino | Isopropyl |
| 2-hydroxyethylamino | 3,4-dihydroxybenzylamino | Isopropyl |
| 3 -hydroxypropylamino | 3,4-dihydroxybenzylamino | Isopropyl |
| (R)-1-(hydroxymethyl) | 3,4-dihydroxybenzylamino | Isopropyl |
| propylamino | | |
| [bis-(2-hydroxyethyl)]-amino | 3,4-dihydroxybenzylamino | Isopropyl |
| 2-(1R-isopropyl-2- | 3,4-dihydroxybenzylamino | Isopropyl |
| hydroxyethylamino) | | |
| | [1-(3,4-dihydroxyphenyl)ethyl]amino | Isopropyl |
| Chloro | [1-(3,4-dihydroxyphenyl)ethyl]amino | Isopropyl |
| 2-hydroxyethylamino | [1-(3,4-dihydroxyphenyl)ethyl]amino | Isopropyl |
| 3-hydroxypropylamino | [1-(3,4-dihydroxyphenyl)ethyl]amino | Isopropyl |
| (R)-1-(hydroxymethyl) | [1-(3,4-dihydroxyphenyl)ethyl]amino | Isopropyl |
| propylamino | | |
| [bis-(2-hydroxyethyl)]-amino | [1-(3,4-dihydroxyphenyl)ethyl]amino | Isopropyl |
| 2-(1R-isopropyl-2- | [1-(3,4-dihydroxyphenyl)ethyl]amino | Isopropyl |
| hydroxyethylamino) | | |
| 2-(1R-isopropyl-2- | [1-(3,4-dihydroxyphenyl)ethyl]amino | Methyl |
| hydroxyethylamino) | | |
| 2-(1R-isopropyl-2- | [1-(3,4-dihydroxyphenyl)ethyl]amino | Ethyl |
| hydroxyethylamino) | | |
| 2-(1R-isopropyl-2- | [1-(3,4-dihydroxyphenyl)ethyl]amino | 2- |
| hydroxyethylamino) | | hydroxyethyl |
| | [N-(2-(3,4-dihydroxyfenyl)ethyl)-N- | Isopropyl |
| | methyl]amino | |
| Chloro | [N-(2-(3,4-dihydroxyfenyl)ethyl)-N- | Isopropyl |
| | methyl]amino | |
| 2-hydroxyethylamino | [N-(2-(3,4-dihydroxyfenyl)ethyl)-N- | Isopropyl |
| | methyl]amino | |
| 3-hydroxypropylamino | [N-(2-(3,4-dihydroxyfenyl)ethyl)-N- | Isopropyl |
| | methyl]amino | |
| (R)-1-(hydroxymethyl) | [N-(2-(3,4-dihydroxyfenyl)ethyl)-N- | Isopropyl |
| propylamino | methyl]amino | |
| (bis-(2-hydroxyethyl)]-amino | [N-(2-(3,4-dihydroxyfenyl)ethyl)-N- | Isopropyl |
| | methyl]amino | |
| 2-(1R-isopropyl-2- | [N-(2-(3,4-dihydroxyfenyl)ethyl)-N- | Isopropyl |
| hydroxyethylamino) | methyl]amino | |
| 2-(1R-isopropyl-2- | [N-(2-(3,4-dihydroxyfenyl)ethyl)-N- | Methyl |
| hydroxyethylamino) | methyl]amino | |
| 2-(1R-isopropyl-2- | [N-(2-(3,4-dihydroxyfenyl)ethyl)-N- | Ethyl |
| hydroxyethylamino) | methyl]amino | |
| 2-(1R-isopropyl-2- | [N-(2-(3,4-dihydroxyfenyl)ethyl)-N- | 2- |
| hydroxyethylamino) | methyl]amino | hydroxyethyl |

### Example 3

6-(3,4-dihydroxybenzylamino)-8-bromo-9-isopropylpurine

1 mmol of 6-(3,4-dihydroxybenzylamino)-9-isopropylpurine was treated with a 2-fold excess of bromide. The product was stirred and heated with 3-fold excess of KCN in 4 mL dimethylformamide to 60° C. for 20 hours. The product was dissolved in 5 mL of 1,3-diaminopropane and the mixture was heated to 150° C. for 10 hours. The excess of diamine was evaporated at 0.1 Torr and the remainder was purified on silica gel column.

### Example 4

6-[N-(2-(3,4-dihydroxyfenyl)ethyl)-N-methyl]amino-8-hydroxyethylamino-9-isopropylpurine

1 mmol of 6-chloropurine was alkylated with isopropylbromide in DMF as described in Example 2. The product 6-chloro-9-isopropylpurine was brominated in acetic acid as described in Example 3. After purification on silica gel column, the product 6-chloro-9-isopropyl-8-bromopurine was transferred to 0.6-[N-(2-(3,4-dihydroxyfenyl)ethyl)-N-methyl]amino-8-hydroxyethylamino-9-isopropylpurine.

### Example 5

Preparation of Affinity Sorbent

For the preparation of 2-(2-aminopropylamino)-6-[N-(2-(3,4-dihydroxyfenyl)ethyl)-N-methyl]amino-8-bromo-9-isopropylpurine epoxy activated Sepharose 6B affinity matrix, freeze-dried epoxy activated Sepharose 6B (Pharmacia LKB, Piscataway, N.J.) was chosen for the coupling reaction due to its ability to form an ether bond between a hydroxyl-containing ligand and the epoxide group on the Sepharose. The gel was swollen according to the manufacturer's instructions, (100 mg) of any one of the purine derivatives according to the invention was dissolved in 1 ml coupling solution (1.2:1, v/v, DMF, 0.1N NaOH) and mixed with 0.5 ml of swollen gel at pH 10-11 for 72 h at room temperature with gentle agitation. Excess reactive groups were blocked with 1M ethanolamine for 4 hours at 50° C. and the gel slurry was poured into 1-ml syringe columns. The resin was activated with three alternating cycles of twenty column volumes each of pH 4.0 (0.1M acetate, 0.5 M NaCl) and pH 8.0 (0.1M tris-HCl, 0.5 M NaCl) buffers followed by twenty column volumes of reaction buffer (20 mM HEPES, pH 7.3, 10 mM MgCl₂₁ 15 mM glycerophosphate, 0.5 mM sodium orthovanadate, 0.5 mM EGTA). The column was stored at 4° C. in reaction buffer containing 0.1 sodium azide, and regenerated prior to each use with alternating cycles of low and high pH as described above.

The Sf9 insect cell lysate (500 μg protein in 1-ml reaction buffer) was passed over the affinity column matrix five times and the flow through was saved (unbound material). The matrix was then washed three times with 1 ml reaction buffer (wash 1-3) and then three times each with reaction buffer containing 0.5M NaCl (eluate 1-3). The coupled proteins were eluted at low pH (pH 4.0, 0.1M acetate, 0.5M NaCl) as described above and aliquots (20 μl from 1 ml) of each sample were assayed for their ability to phosphorylate histone H1 and other substrate proteins as described in Example 12. The presence of CDK complexes was also determined by SDS-PAGE.

### Example 6

Cdk Inhibition Assays

Cyclin-dependent kinases (p34^{cdc2}, p33^{cdk2}, p33^{cdk4} and cyclins (cyclin B, E and DI) are produced in Sf9 insect cells coinfected with appropriate baculoviral constructs. The cells are harvested 68-72 hrs post infection in lysis buffer for 30 min on ice and the soluble fraction is recovered by centrifugation at 14.000 g for 10 min. The protein extract is stored at −80° C.

Rb-GST is produced using an E. coli expression system, containing a sequence encoding the C terminus of retinoblastoma protein (aminoacids 773-928), which is known to be phosphorylated by p33^{cdk4} kinase. The fusion protein is purified on glutathione-agarose beads. Lysis buffer: 50 mM Tris pH 7.4, 150 mM NaCl, 5 mM EDTA, 20 mM NaF, 1% Tween, 1 mM DTT, 0.1 mM PMSF, leupeptine, aprotonine.

Enzyme Inhibition Assays

To carry out experiments on kinetics under linear conditions, the final point test system for kinase activity measurement is used. The kinase is added to the reaction mixture in such a way that linear activity is obtained with respect to the concentration of enzyme and with respect to time.

The p34^{cdc2} and p33^{cdk2} kinase inhibition assays involve the use of 1 mg/ml histone H1 (Sigma, type III-S) in the presence of 15 μM [γ-³²P)ATP (500-100 cpm/pmol) (Amersham) in a final volume of 20 μl. Inhibition of p33^{cdk4} kinase is determined with Rb-GST (0.2 mg/ml) as the substrate. Kinase activity is determined at 30° C. in kinase buffer.

Tested compounds are usually dissolved to 100 mM solutions in DMSO, the final concentration of DMSO in reaction mixture never exceeds 1%. The controls contain suitable dilutions of DMSO. After 10 min, the addition of 3×SDS sample buffer stops the incubations. Phosphorylated proteins are separated electrophoretically using 12.5% SDS polyacrylamide gel. The measurement of kinase activity is done using digital image analysis. The kinase activity is expressed as a percentage of maximum activity, the apparent inhibition constants are determined by graphic analysis. Kinase buffer: 50 mM Hepes pH 7.4, 10 mM MgCl₂₁ 5 mM EGTA, 10 mM 2-glycerolphosphate,

1 mM NaF, 1 mM DTT.

Table 3 shows the results of the inhibitory activity of the purine derivatives according to the invention against CDC2 and IκB-α. The 2,6,9-trisubstituted purine derivatives showed marked inhibitory activity in in vitro kinase assays. Modification of 2,6,9-trisubstituted purines by a catechol-like substituent at R6 leads to an increase in cdk inhibitory activity of the tested compounds.

TABLE 3
Kinase-inhibitory activity of 2, 6, 9-
trisubstituted purine derivatives according to the
invention

| SUBSTITUENT | | | CDC2 | | IκB-α | |
|---|---|---|---|---|---|---|
| C2 | N6 | N9 | IC₅₀ (μM) | | IC₅₀ (μM) | |
| 2- | 3,4-dihydroxybenzylamino | Methyl | 4.2 | | 10.8 | |
| hydroxyethylamino | | | | | | |
| 2- | [(R,S)-(1-phenyl-2- | Methyl | 5.6 | | 13.7 | |
| hydroxyethylamino | hydroxyethyl)amino] | | | | | |
| 2- | [N-(2-(3,4-dihydroxyfenyl)ethyl)- | Methyl | 10.2 | | 25.4 | |
| hydroxyethylamino | N-methyl]amino | | | | | |
| (R)-1- | 3,4-dihydroxybenzylamino | Isopropyl | 0.32 | | 1.25 | |
| (hydroxymethyl) | | | | | | |
| propylamino | | | | | | |
| Hexylamino | [N-(2-(3,4-dihydroxyfenyl)ethyl)- | Isopropyl | 2.6 | | | |
| | N-methyl]amino | | | | | |
| (R,S)-2- | [(R,S)-(1-phenyl-2- | Isopropyl | 0.6 | | | |
| hydroxypropyl | hydroxyethyl)amino] | | | | | |
| amino | | | | | | |
| 2- | [(R,S)-(1-phenyl-2- | Isopropyl | 1.4 | | | |
| hydroxyethylamino | hydroxyethyl)amino] | | | | | |
| (R)-1- | [(R,S)-(1-phenyl-2- | Isopropyl | 0.67 | | 2.15 | |
| (hydroxymethyl) | hydroxyethyl)amino] | | | | | |
| propylamino | | | | | | |
| (R)-1- | [N-(2-(3,4-dihydroxyfenyl)ethyl)- | Isopropyl | 0.95 | | 3.20 | |
| (hydroxymethyl) | N-methyl]amino | | | | | |
| propylamino | | | | | | |
| 2-(1R-isopropyl-2- | 3,4-dihydroxybenzylamino | Isopropyl | 250 | nM | 320 | nM |
| hydroxyethylamino) | | | | | | |
| 2-(1R-isopropyl-2- | [(R,S)-(1-phenyl-2- | Isopropyl | 540 | nM | 670 | nM |
| hydroxyethylamino) | hydroxyethyl)amino] | | | | | |
| 2-(1R-isopropyl-2- | [N-(2-(3,4-dihydroxyfenyl)ethyl)- | Isopropyl | 390 | nM | 460 | nM |
| hydroxyethylamino) | N-methyl]amino | | | | | |

### Example 7

Modulation of the Activity or Signal Transduction of β-Adrenergic/Purinergic Receptors

Rat C6 glioma (ATCC N° CCL107) was cultivated in monolayer in serum-free chemically defined medium containing Ham's F10/minimal essential medium (1:1 vol/vol), 2 mM L-glutamine, 1% (vol/vol) minimal essential medium vitamins (100×), 1% (vol/vol) minimal essential medium nonessential amino acids (100×), 100 U/ml penicillin, 100 μg/ml streptomycin and 30 nM sodium selenite. Incubation was at 37° C. in a humidified atmosphere. Assays were performed in the logaritmic growth phase at a density of 2.5×10⁵ cells/cm². Intracellular cAMP synthesis was induced by addition of 5 μM (−) isoproterenol. After 30 min incubation at 37° C. the medium was removed and the cellular amount of cAMP determined using the cAMP-enzyme immunoassay kit of Amersham. The IC₅₀ is determined from a dose-response curve in duplicate. The effect of seven purine-analogs was measured after simultaneous addition with isoproterenol.

TABLE 4
Modulation of the activity of β-adrenergic
receptors by substituted purine derivatives

| Analog | C2 | N6 | N9 | Effect | I₅₀ (μM) |
|---|---|---|---|---|---|
| P23 | Hexylamino | [(R,S)-(1-phenyl-2- | Isopropyl | inhibition | 15 ± 2 |
| | | hydroxyethyl)amino] | | | |
| P12 | 3-aminopropylamino | Benzylamino | Isopropyl | inhibition | 45 ± 5 |
| P30 | (1-hydroxymethyl-2- | Benzylamino | Isopropyl | inhibition | 45 ± 5 |
| | methyl)propylamino | | | | |
| P19 (50 μM) | (R)-(1-hydroxymethyl) | 4-hydroxybenzyl | Isopropyl | 1.8-fold activation | |
| | propylamino | amino | | | |
| P29 (50 μM) | (R)-(1-hydroxymethyl) | 3-hydroxybenzyl | Isopropyl | 1.7-fold activation | |
| | propylamino | amino | | | |
| P28 (50 μM) | 2-aminoethylamino | Benzylamino | Isopropyl | 1.3-fold activation | |
| P38 | (S)-(1-hydroxymethyl) | (R)-hydroxy-1- | Isopropyl | inactive | |
| | propylamino | phenylethylamino | | | |
| P39 | 2-hydroxypropylamino | (R)-hydroxy-1- | Isopropyl | inactive | |
| | | phenylethylamino | | | |

As P2Y₁-like and A2 purinergic receptors, negatively and positively coupled to adenylate cyclase respectively, are present on rat C6 glioma the modulation of the synthesis of cAMP may be due to inhibition of the activation of β-adrenergic receptors by isoproterenol or to activation of purinergic receptors.

### Example 8

Effect of the Novel Compounds on the Proliferation of Hematopoietic Cells

Cell Separation and Cell Cultures:

Cell lines: Human leukemic cell lines were obtained from the American Type Culture Collection (ATCC, Rockville, Md., USA). They were cultured in Iscove's Modified Dulbecco's Medium (IMDM) supplemented with 10% heat inactivated fetal calf serum (FCS), 200 U/ml penicillin, 200 μg/ml streptomycin and 1 μg/ml amphotericin B. Cells were cultured in a 5% CO₂-95% air fully humidified incubator. For effects on clonogenic output, 1000 cells/well were plated in duplicate in methylcellulose (0.9%), supplemented with 20% FCS and cultured for 14 days.

Peripheral Blood Mononuclear Cells (PBMC):

Human peripheral blood mononuclear cells were isolated by density gradient (Ficoll-Hypaque) (LSM, ICN Biomedicals Inc.). PBMC were stimulated with 5 μg/ml phytohemaglutinin A (PHA) (Sigma) during 24-48 hours in IMDM supplemented with 10% FCS (IMDM/10% FCS) at 37° C. After washing off the PHA, PBMC were incubated with interleukin-2 (IL-2) (10 U/ml) (Genzyme).

Adult Bone Marrow (ABM) Cells:

Bone marrow samples were obtained by sternal puncture from hematologically normal donors undergoing cardiac surgery, after obtaining informed consent. Cells were collected in IMDM supplemented with 10% FCS and 100 U/ml heparin and separated by density gradient as mentioned for PBMC. After washing, cells were resuspended in IMDM/10% FCS and were sorted on a FACStar (Becton Dickinson, Erembodegem, Belgium).

CD34⁺ Cell Sorting:

ABM cells (10⁷ cells/ml) were incubated with 43A1 supernatant in a 1/10 dilution for 15 minutes at 4° C. The supernatant of the 43A1 hybridoma (immunoglobulin (Ig)G3) was kindly donated by Dr. H. J. Buhring (University of Tubingen, Germany) and was used as a source of anti-CD34 antibodies. Then cells were washed twice in IMDM and incubated with FITC-conjugated rabbit anti-mouse Ig (1/50 dilution) for 15 minutes at 4° C. After washing twice in IMDM, CD34⁺ were sorted on a FACStarPlus cell sorter equipped with an water-cooled argon ion laser (INNOVA Enterprise Ion Laser) with multiple wave lengths including UV (488 nm).

Myeloid Colony-Forming Unit (CFU) Assays:

Direct myeloid colony formation of CD34⁺ cells was assessed in a CFU assay. These assays were initiated with 500 cells per well and plated in duplicate in methylcellulose (0.9%) supplemented with 20 a FCS, 1% bovine serum albumin (BSA), 10⁻⁵ M mercaptoethanol and 10 vol. % of conditioned medium of the 5637 bladder carcinoma cell line (containing G-CSF and GM-CSF), 2 U/ml erythropoietin and 30 U/ml Interleukin 3 (IL-3). After 14 days of culture at 37° C. in 7.5 a 02 and 5% CO₂ in a fully humidified incubator, these cultures were scored with the microscope for colony formation. The following colony types were scored: myeloid colonies: macrophage (CFU-M), granulocyte (CFU-G), and granulocyte-macrophage (CFU-GM); erythroid colonies (BFU-E (burst-forming units, erythroid) and CFU-E); and mixed erytroid-myeloid colonies (CFU-Mix).

CD34⁺CD38⁻ Cell Sorting:

ABM cells (10⁷ cells/ml) were incubated with 43A1 hybridoma supernatant at a 1/10 dilution for 20 minutes at 4° C. After washing twice in IMDM, the cells were incubated with FITC-conjugated rabbit anti-mouse IgG (1/40 dilution) for 20 min at 4° C. After washing twice, cells were incubated for 10 minutes with 5 μg mouse Ig and for 15 minutes with anti-CD38-PE (20 μl/10⁶ cells) After washing twice in IMDM, the cells were sorted on a FACStarPlus cell sorter equipped with an water-cooled argon ion laser (INNOVA Enterprise Ion Laser) with multiple wave length outputs including UV (488 nm). Cells with low-to-medium forward and low side scatter, highly positive green (CD34) fluorescence, and an orange (CD38) fluorescence signal lower than the mean fluorescence of cells labeled with an irrelevant isotype-matched control antibody+2 standard deviations were retained as CD34⁺CD38⁻ cells and used in pre-CFU assays.

Pre-CFU:

Pre-CFU assays were initiated by performing liquid cultures of CD34⁺CD38⁻ cells in duplicate in 96-well flat-bottomed plates in IMDM/10% FCS, 1% bovine serum albumin (BSA), 100 U/ml IL-1, 200 U/ml IL-6, 30 U/ml IL-3 and 100 ng/ml stem cell factor (SCF). Pre-CFU cultures were initiated with 500 CD34⁺CD38⁻ cells/well (200 μl). After 14 days of culture at 37° C. in 7.5% O₂ and 5% CO₂ in a fully humidified incubator, the number of cells in each well was counted. Subsequently, the cells were harvested, washed three times in IMDM/10% FCS, and plated in duplicate at 500 cells/well (1000 μl) in secondary methylcellulose CFU cultures as described for CFU assays.

Effect of New Compounds on Cell Proliferation

Cells were plated at 10000 per well in 200 μl IMDM medium and incubated with 0-50 μM of the compounds of the invention (Table 5), by addition of the drugs directly to the culture medium and incubated at 37° C. for 96 hours. Absolute cell number was determined by addition of a known concentration of Fluoresbrite microspheres (FITC) (Polysciences, Inc.). The absolute number of cells per well was calculated as: {(total number of beads added per well)/(number of beads measured)}×(number of measured cells in the gate of interest). All analyses were performed with a FACScan (BD), using CELLQuest software (Becton Dickinson).

The response of the myeloid leukemia KG1 and T-lymphocyte leukemia Molt3 cell lines to the cytostatic effect of the novel compounds was determined using the above-mentioned standardized bead suspension that was used to determine the absolute cell number by flow cytometric (FCM) measurement. Cells were grown in the presence of increasing concentrations of the compounds of the invention. After 96 hours of culture the concentration at which cell growth was inhibited by 50%—the 50% inhibitory concentration or IC₅₀—was calculated from dose-response curves (FIG. 1; Table 5).

Different response patterns were seen for the novel compounds tested, with IC₅₀ ranging from 7 μM to >50 μM for KG1 and from 9 μM to >50 μM for Molt3.

Clonogenic output of KG1 was tested in methylcellulose with 25 μM of some of the novel compounds. Colony output vs. control cultures without novel compound, was 47% for P3, 16% for P16, 19% for P23, 8% for P27 and 0% for P28.

The novel compounds were tested for cytotoxicity on PHA-stimulated lymphocytes (PBMC), as one of the normal counterparts of the cell lines. Cells were grown for 96 hours in the presence of IL-2 and different concentrations of the novel compounds and cell number was counted on the flow cytometer. The IC₅₀ values are shown in Table 5.

TABLE 5
Tested purine derivatives: structural names and
IC₅₀ values on the different cell culture systems
(lymphocytes, KG1, Molt3, CFU and pre-CFU) and on CDC2
activity in cell free system.

| | | | KGI | | CFU | | |
|---|---|---|---|---|---|---|---|
| | | Lymphocytes | IC₅₀ | Molt3 | IC₅₀ | Pre-CFU | CDC2 |
| Structural name | No. | IC₅₀ (μM) | (μM) | IC₅₀ (μM) | (μM) | IC₅₀ (μM) | IC₅₀ (μM) |
| 2-(2-hydroxyethylamino)-6-(2- | P3 | 51 | 51 | 51 | >50 | >25 | 65 |
| isopentenylamino)-9-methylpurine | | | | | | | |
| 2-(3-hydroxypropylamino)-6-benzylamino- | P10 | 10 ± 0.4 | 35 ± 4.5 | 44 ± 2.8 | | | 1 |
| 9-isopropylpurine | | | | | | | |
| 2-(3-aminopropylamino)-6-benzylamino-9- | P12 | 8 ± 1.2 | 16 ± 1.7 | 19.3 ± 2.3 | 37 | 20 | 21 |
| isopropylpurine | | | | | | | |
| 2-(methylthio)-6-[N-(3,4-dihydroxybenzyl- | P16 | 26.5 ± 2.8 | 23 ± 1.2 | 37 ± 7.9 | >50 | 16 | >200 |
| α-methyl]amino-9-isopropylpurine | | | | | | | |
| 2-(hexylamino)-6-[N-(3,4-dihydroxybenzyl- | P17 | 18 ± 2.8 | 35.3 ± 5.7 | 24 ± 6.2 | | | 1 |
| α-methyl]amino-9-isopropylpurine | | | | | | | |
| 2-(3-hydroxypropylamino)-6[N-(3,4- | P18 | 14.9 ± 2.9 | 18.7 ± 0.3 | 23 ± 2.8 | | | 2.6 |
| dihydroxybenzyl-α-methyl]amino-9- | | | | | | | |
| isopropylpurine | | | | | | | |
| 2-(1-ethyl-2-hydroxyethylamino)-6)-9- | P19 | 6 | 11 ± 1.3 | 15 ± 2 | | | 1 |
| isopropylpurine | | | | | | | |
| 2-(3-hydroxypropylamino)-6[N-(3,4- | P20 | 35 ± 7.3 | 51 | 51 | | | |
| dihydroxybenzyl-α-methyl]amino-9- | | | | | | | |
| isopropylpurine | | | | | | | |
| 2-(morpholino)-6-(1-phenyl-2- | P21 | 40 ± 7.7 | 48 ± 1.5 | 50 ± 1.3 | | | 6.5 |
| hydroxyethylamino)-9-methylpurine | | | | | | | |
| 2-(2-hydroxyethylthio)-6-(1-phenyl-2- | P22 | 11 ± 1.5 | 13 ± 1.4 | 17 ± 1.7 | | | 3.8 |
| hydroxyethylamino)-9-isopropylpurine | | | | | | | |
| 2-(hexylamino)-6-(1-phenyl-2- | P23 | 15 ± 2 | 8 ± 0.6 | 16 ± 0.8 | >50 | >25 | 60 |
| hydroxyethylamino)-9-isopropylpurine | | | | | | | |
| 2-(5-cyanopentyl)-6-[(R,S)-(1-phenyl-2- | P24 | 33 ± 4.4 | 42 ± 4.8 | 42.5 ± 6 | | | 6.3 |
| hydroxyethyl)amino]-9-isopropylpurine | | | | | | | |
| 2-(3-hydroxypropylamino)-6-benzylthio-9- | P25 | 18 ± 1.6 | 42 ± 5 | 19 ± 2 | | | >100 |
| isopropylpurine | | | | | | | |
| 2-(2,3-dihydroxypropylamino)-6-[(R,S)-(1- | P26 | 20 ± 1 | 20 ± 1 | 23.7 ± 3.4 | | | >100 |
| phenyl-2-hydroxyethyl)amino]-9- | | | | | | | |
| isopropylpurine | | | | | | | |
| 2-(3-hydroxypropylamino)-6-[(R,S)-(1- | P27 | 8 ± 0.5 | 7 ± 2 | 14 ± 1.5 | >50 | >25 | >100 |
| phenyl-2-hydroxyethyl)amino]-9- | | | | | | | |
| isopropylpurine | | | | | | | |
| 2-(3-aminoethylamino)-6-(3,3- | P28 | 9 ± 2.2 | 16 ± 1.3 | 14 ± 1.8 | 8.5 | 3.5 | 1.5 |
| dihydroxybenzyl)amino-9-isopropylpurine | | | | | | | |
| 2-(1-ethyl-2-hydroxyethylamino)-6(3,4- | P29 | 9 ± 6.6 | 16.5 ± 1 | 9 ± 1.2 | | | 10 |
| dihydroxybenzylamino)-9-isopropylpurine | | | | | | | |

Comparison between normal PHA-stimulated lymphocytes and hematopoietic cell lines shows that lymphocytes are more sensitive to some of the novel compounds than cell lines, with the exception of P23, that was significantly more effective on KG1 than on normal-PBMC (FIG. 2). To determine reversibility of the effects of the new compounds, cells were plated at 10,000 per well and exposed to 0-50 μM of the composed, either continuously for 7 days (control) or only for 6 hours. In the latter case, cells were washed after 6 hours in phosphate-buffered-saline (PBS) (Life Technologies) and reseeded into drug-free medium for 7 days. After 7 days (168 hours), cell number was assayed by flow cytometry for both culture conditions. When KG1 cells were exposed continuously to compounds P12, P27 and P28 the IC₅₀ were respectively 16±1.7 μM; 7±2 μM and 16±1.3 μM (Table 5). However, if cells were washed free of novel compounds after 6 hours of exposure to P12, P27 and P28, there was (some) recovery of cell number as compared to control without novel compounds (FIG. 1a,b,c).

CD34⁺ hematopoietic progenitors (HPC) from adult bone marrow were isolated and investigated for their response to the novel compounds. CD34⁺ cells were grown in a methylcellulose system in the presence of increasing concentrations of compounds. After 14 days, colonies were microscopically scored and IC₅₀ concentrations were calculated from the dose-response patterns of total colony output (Table 5). P3, P16, P23 and P27 have low or no inhibitory activity on progenitors with IC₅₀>50 μM. P28 has potent inhibitory activity with a IC₅₀ of 8.5 μM, P12 has intermediate effect with an IC₅₀ of 37 μM.

Clonogenic output of CD34⁺ HPC was also scored differentially. P3 and P16 caused no significant difference in the output of the different types of myeloid colonies. Culture with P12, P23, P27 and P28 resulted in significantly lower colony output for CFU-E, CFU-G and CFU-M, with an exception for P27 where CFU-M were not significantly decreased. No significant difference was seen for CFU-GM and CFU-MIX for tested compounds. Control semi-solid cultures with DMSO were not significantly different from the control cultures.

Pre-CFU were cultured starting from adult bone marrow CD34⁺CD38⁻ cells, that had been isolated using the FCM cell sorter. Novel compounds were added at different concentrations to the primary 14 day liquid culture. IC₅₀ was calculated from dose-response curves from total clonogenic output after secondary methylcellulose culture (Table 5). Effects were in a range similar to those on CFU with an exception for P16 that was more active on pre-CFU than on CFU.

### Example 9

Antimitotic Activities of CDK Inhibitors

Metaphase-arrested Xenopus egg extracts were prepared as described previously by Blow “J. Cell Biol.” 1993, 122:993 and stored in liquid nitrogen. Demembranated Xenopus sperm nuclei were prepared as described by Blow & Laskey “Cell” 1986;47:577. After thawing, extracts were supplemented with 25 mM phosphocreatine, 5 μg/ml creatine phosphokinase, 250 μg/ml cycloheximide, (α-³²P]dATP (for DNA synthesis assays). Demembranated sperm nuclei were added to a final sperm concentration of 3 ng/μl DNA extract and CDK inhibitor tested was then added at different concentrations. M-phase promoting factor inhibition by different CDK inhibitors was monitored 1.5 h after addition by assessing the amount of sperm nuclei that had been assembled into interphase nuclei, possessing a complete phase-dense nuclear envelope. DNA synthesis was assessed by releasing extract into interphase by the addition of 0.3 mM CaCl₂ and measuring the total amount of [α-³²P]dATP incorporation after 3 h by TCA co-precipitation.

At concentrations of cdk inhibitors (see Table 6) ranging from 0.1-2 μM, chromosomes remained highly condensed and no nuclear envelope was visible. At 4-6 μM and higher concentrations, interphase nuclei appeared with partially decondensed chromatin and an intact nuclear envelope. Replication was significantly inhibited at 1-5 μM CDK inhibitors tested. For the inhibition effect to become detectable, the first 15-min incubation of the interphase extract is probably sufficient.

TABLE 6
Antimitotic activities of 2, 6, 9-trisubstituted
purine derivatives

| | | | Inhibition of MPF | Inhibition of DNA |
|---|---|---|---|---|
| SUBSTITUENT | | | activity | synthesis |
| C2 | N6 | N9 | IC₅₀ (μM) | IC₅₀ (μM) |
| Hydroxypropylamino | 3,4-dihydroxybenzylamino | isopropyl | 3.6 | 4.2 |
| Hydroxypropylamino | [(R,S)-(1-phenyl-2-hydroxyethyl)amino] | isopropyl | 1.5 | 1.4 |
| Hydroxypropylamino | [N-(2-(3,4-dihydroxyfenyl)ethyl)-N- | isopropyl | 5.6 | 6.7 |
| | methyl]amino | | | |
| (R)-1-(hydroxyethyl) | 3,4-dihydroxybenzylamino | isopropyl | 2.8 | 3.5 |
| propylamino | | | | |
| (R)-1-(hydroxyethyl) | [(R,S)-(1-phenyl-2-hydroxyethyl)amino] | isopropyl | 0.9 | 1.25 |
| propylamino | | | | |
| (R)-1-(hydroxymethyl) | [N-(2-(3,4-dihydroxylfenyl)ethyl)-N- | isopropyl | 1.12 | 1.3 |
| propylamino | methyl]amino | | | |

### Example 10

In vitro Cytotoxic Activity of Novel Compounds of the Invention

One of the parameters used as the basis for colorimetric assays is the metabolic activity of viable cells. For example, a microtiter assay, which uses the tetrazolium salt MTT, is now widely used to quantitate cell proliferation and cytotoxicity. For instance, this assay is used in drug screening programs and in chemosensitivity testing. Because only metabolically active cells cleave tetrazolium salts, these assays detect viable cells exclusively. In the case of MTT assay, yellow soluble tetrazolium salt is reduced to coloured water-insoluble formazan salt. After it is solubilized, the formazan formed can easily and rapidly be quantified in a conventional ELISA plate reader at 570 nm (maximum absorbance). The quantity of reduced formazan corresponds to number of vital cells in the culture. Human T-lymphoblastic leukemia cell line CEM; promyelocytic HL-60 and monocytic U937 leukemias; breast carcinoma cell lines MCF-7, BT549, MDA-MB-231; glioblastoma U87MG cells; cervical carcinoma cells HELA; sarcoma cells U20S and Saos2; hepatocellular carcinoma HepG2; mouse immortalized bone marrow macrophages B2.4 and B10A.4; P388D1 and L1210 leukemia; B16 and B16F10 melanomas were used for routine screening of the compounds according to the invention. The cells were maintained in Nunc/Corning 80 cm² plastic tissue culture flasks and cultured in cell culture medium (DMEM with 5 g/l glucose, 2 mM glutamine, 100 U/ml penicillin, 100 μg/ml streptomycin, 10% fetal calf serum and sodium bicarbonate).

The cell suspensions that were prepared and diluted according to the particular cell type and the, expected target cell density (2500-30000 cells per well based on cell growth characteristics) were added by pipette (80 μl) into 96-well microtiter plates. Inoculates were allowed a pre-incubation period of 24 hours at 37° C. and 5% CO₂ for stabilisation. Four-fold dilutions of the intended test concentration were added at time zero in 20 μl aliquots to the microtiter plate wells. Usually, test compound was evaluated at six 4-fold dilutions. In routine testing, the highest well concentration was 266.7 μM, but it can be the matter of change dependent on the agent. All drug concentrations were examined in duplicates. Incubations of cells with the test compounds lasted for 72 hours at 37° C., in 5% CO₂ atmosphere and 100% humidity. At the end of incubation period, the cells were assayed by using the MTT. Ten microliters of the MTT stock solution were pipetted into each well and incubated further for 1-4 hours. After this incubation period, formazan was solubilized by addition of 100 μl/well of 10% SDS in water (pH=5.5) followed by further incubation at 37° C. overnight. The optical density (OD) was measured at 540 nm with the Labsystem iEMS Reader MF (UK). The tumour cell survival (TCS) was calculated using the following equation: TCS=(OD_{drug exposed well}/mean OD_{control wells})×100%. The TCS₅₀ value, the drug concentration lethal to 50% of the tumour cells, was calculated from the obtained dose response curves.

The cytoxicity of novel compounds was tested on panel of cell lines of different histogenetic and species origin (Table 7). Equal activities were found in all tumour cell lines tested. Notably, identical effectiveness of purine derivatives was also found in cell lines bearing various mutations or deletions in cell cycle associated proteins, e.g. HL-60, BTS49, Hela, U20S, MDA-MB231, and Saos2. This indicates that these substances are equally effective in tumours with various alterations of tumour suppressor genes, namely p53, Rb, etc. Importantly, this observation distinguishes the purine derivatives of the invention from flavopiridol and related compounds, as their biological activity is dependent on p53 status.

TABLE 7
Cytotoxicity of compounds of the invention for
different cancer cells.

| SUBSTITUENT | | | CEM | B16 |
|---|---|---|---|---|
| C2 | C6 | N9 | IC₅₀ (μM) | IC₅₀ (μM) |
| (R)-2-hydroxypropylamino | [(R,S)-(1-phenyl-2-hydroxyethyl)amino] | isopropyl | 70 | 74.6 |
| (S)-2-hydroxypropylamino | [(R,S)-(1-phenyl-2-hydroxyethyl)amino] | isopropyl | 8 | 5.6 |
| Hexylamino | [(R,S)-(1-phenyl-2-hydroxyethyl)amino] | isopropyl | 34 | 36.8 |
| 2-hydroxyethylamino | [(R,S)-(1-phenyl-2-hydroxyethyl)amino] | isopropyl | 18 | 19 |
| (R)-1-(hydroxymethyl) propylamino | [(R,S)-(1-phenyl-2-hydroxyethyl)amino] | isopropyl | 15 | 13.5 |
| (S)-1-(hydroxymethyl) propylamino | [(R,S)-(1-phenyl-2-hydroxyethyl)amino] | isopropyl | 20 | 24 |
| (R)-2-hydroxypropylamino | 3,4-dihydroxybenzylamino | isopropyl | 35 | 41 |
| (S)-2-hydroxypropylamino | 3,4-dihydroxybenzylamino | isopropyl | 12 | 10 |
| Hexylamino | 3,4-dihydroxybenzylamino | isopropyl | 22 | 21 |
| 2-hydroxyethylamino | 3,4-dihydroxybenzylamino | isopropyl | 35 | 32 |
| (R)-1-(hydroxymethyl) propylamino | 3,4-dihydroxybenzylamino | isopropyl | 8 | 9 |
| (S)-1-(hydroxymethyl) propylamino | 3,4-dihydroxybenzylamino | isopropyl | 51 | 53 |
| (R)-2-hydroxypropylamino | [N-(2-(3,4-dihydroxyfenyl)ethyl)-N- | isopropyl | 13 | 14 |
| | methyl]amino | | | |
| (S)-2-hydroxypropylamino | [N-(2-(3,4-dihydroxyfenyl)ethyl)-N- | isopropyl | 43 | 40 |
| | methyl]amino | | | |
| Hexylamino | [N-(2-(3,4-dihydroxyfenyl)ethyl)-N- | isopropyl | 33 | 32 |
| | methyl]amino | | | |
| 2-hydroxyethylamino | [N-(2-(3,4-dihydroxyfenyl)ethyl)-N- | isopropyl | 27 | 26 |
| | methyl]amino | | | |
| (R)-1-(hydroxymethyl) propylamino | [N-(2-(3,4-dihydroxyfenyl)ethyl)-N- | isopropyl | 6 | 6 |
| | methyl]amino | | | |
| (S)-1-(hydroxymethyl) propylamino | [N-(2-(3,4-dihydroxyfenyl)ethyl)-N- | isopropyl | 36 | 34 |
| | methyl]amino | | | |
| 2-(1R-isopropyl-2- | (N-(2-(3,4-dihydroxyfenyl)ethyl)-N- | isopropyl | 8 | 9 |
| hydroxyethylamino) | methyl]amino | | | |

### Example 11

Novel Compounds Induce Apoptosis in Tumour Cells

To analyse the mechanisms of induced cytotoxicity by novel compounds, it is important to distinguish apoptosis from the other major form of cell death, necrosis. First, at the tissue level, apoptosis produces little or no inflammation, since the neighbouring cells, especially macrophages, rather than being released into the extracellular fluid, engulf shrunken portions of the cell. In contrast, in necrosis, cellular contents are released into the extracellular fluid, and thus have an irritant affect on the nearby cells, causing inflammation. Second, at the cellular level, apoptotic cells exhibit shrinkage and blebbing of the cytoplasm, preservation of structure of cellular organelles including the mitochondria, condensation and margination of chromatin, fragmentation of nuclei, and formation of apoptotic bodies, thought not all of these are seen in all cell types. Third, at the molecular level, a number of biochemical processes take an important role in induction of apoptosis. However, majority of them is not well understood, and they result in activation of proteases and nucleases, which finally destruct key biological macromolecules—proteins and DNA. For detection of apoptotic versus necrotic mode of cell death, two independent methods were employed: assessment of morphology by fluorescence microscopy and analysis of DNA fragmentation by flow cytometry using the TUNEL technique.

Determination of Apoptosis and Cell Cycle Distribution

Microscopy:

Nuclear morphology of the cells was analysed with the fluorochromes Hoechst 33342 (λ_{Ex} max 346 nm; λ_{Em} max 460 nm) (Sigma) prepared in phosphate-buffered saline (PBS at 0.1 mg/ml, added to the culture medium at a final concentration of 2 μg/ml and ethidium bromide (EB) (λ_{Ex} max 540 nm; λ_{Em} max 625 nm) (Sigma) prepared in PBS at 100 μg/ml and added to the culture medium at a final concentration of 2 μg/ml (Lizard, 1995). Hundred cells were counted for each sample and percentage of apoptosis was determined.

TdT-Mediated dUTP Nick End Labeling (TUNEL):

Control and novel compound-treated cell cultures were washed with PBS and fixed in 1 buffered formaldehyde (pH 7.4) for 15 minutes on ice. After washing in PBS, cells were permeabilized in 70% cold (−20° C.) ethanol and transferred to 4° C. for at least 1 hour. After rehydratation in PBS, cells were labeled with 50 μl/well TUNEL reaction mixture (Boehringer Mannheim). Cells were incubated in this solution at 37° C. for 40 minutes, washed in PBS and resuspended in 500 μl PBS containing 5 μg/ml EB and 0.1% RNAse. After 30 minutes of incubation at 4° C., green (FITC-dUTP) and red (EB-DNA) fluorescence of individual cells was measured on a FACscan flow cytometer (Gorczyca, 1993). Negative control (fixed and permeabilized cells incubated with 50 μl label solution per well without terminal transferase, instead of TUNEL reaction mixture) and positive control (fixed and permeabilized cells incubated with DNase I (100 μg/ml) that induces DNA strand breaks) were included in each experimental set-up.

Flow cytometric detection of apoptosis combined with DNA staining can identify apoptotic cells and simultaneously evaluate their position in the cell cycle. This technique was used to provide infomation about the possible cell cycle specific initiation of apoptosis by the tested compounds. If more than 2% of apoptotic cells were detected by the TUNEL method and the absolute number of acquired apoptotic events exceeded 2000, DNA content was simultaneously measured. Within the apoptotic population, analysis and calculation of the different cell cycle phases was carried out by an iterative curve fitting procedure (ModFit LT cell cycle analysis sofware from Verity software house, INC).

Pro-Apoptotic Effect of New Compounds

Fluorescence microsopy analysis of apoptosis and necrosis: Cell cultures treated with different doses of novel compounds (“P” number of compounds is as in Table 5 in Example 8) were examined microscopically for apoptosis. Apoptotic cells exhibit a very bright Hoechst 33342 fluorescence, while viable cells display a very faint fluorescence. Late apoptotic cells or secondarily necrotic cells display a fragmented nucleus with bright red ethidium bromide fluorescence. Primary necrotic cells show a red fluorescence and do not have fragmented nuclei. An illustration of these different features can be found in FIG. 3 (Jurkat T-cell line incubated with compound P12).

FIG. 4 shows the result of microscopic examination of KG1 cells incubated with P23, P27 and P28. Viable, apoptotic, necrotic (=primary necrosis, not following apoptosis) and secondarily necrotic cells (=late apoptotis, evolving to necrosis) were scored differentially after 6, 12, 24, 48 and 72 hours of exposure to novel compounds. For the three products a different apoptosis-inducing pattern could be observed. Apoptosis induction occurs fast after incubation with P23 and P28 but slower after incubation with P27.

Flow cytometric detection of apoptosis and cell cycle analysis: The induction of apoptotic death of KG1 cells by the novel compounds was confirmed using the TUNEL reaction technique (FIG. 5). For P23, apoptotic cell number in G₀-G₁ phase of the cell cycle is higher in comparison with the control cells all time points (untreated culture), but no significant differences were detected (FIG. 6a). Incubation of KG1 cell line with P27 results in detectable apoptosis after 24 hours. Percentage of apoptotic cells in G₀-G₁ increases with time, while the number of apoptotic cells in S+G₂/M phases decreases. These differences between control and apoptotic cells were significant after 72 hours (p=0.035). No significant difference was detected between control (without incubation of product) and the non-apoptotic population in the incubated culture as measured by TUNEL (FIG. 6b). Exposure to P28 demonstrates a different time response: apoptosis was already detected after 6 hours of incubation. After 6 and 12 hours of incubation the apoptotic population seems mainly to evolve from S phase. Apoptotic cells in G₀-G₁ phase increase with time after 24 hours. No significant difference was detected between control and the non-apoptotic population in the incubated culture (FIG. 6c).

### Example 12

Immunosuppressive Activity

One of the most important parameters of specific cellular immunity is proliferative response of lymphocytes to antigens or polyclonal mitogens. The majority of normal mammalian peripheral lymphocytes comprise resting cells. Antigens or nonspecific polyclonal mitogens have the capacity to activate lymphoid cells and this is accompanied by dramatic changes of intracellular metabolism (mitochondrial activity, protein synthesis, nucleic acids synthesis, formation of blastic cells and cellular proliferation). Compounds with ability to selectively inhibit lymphocyte proliferation are potent immunosuppressants. A variety of in vitro assays was developed to measure the proliferative response of lymphocytes. The most commonly used is ³H-thymidine incorporation method. During cell proliferation, DNA has to be replicated before the cell is divided into two daughter cells. This close association between cell doublings and DNA synthesis is very attractive for assessing cell proliferation. If labeled DNA precursors are added to the cell culture, cells that are about to divide incorporate the labeled nucleotide into their DNA. Traditionally, those assays usually involve the use of radiolabeled nucleosides, particularly tritiated thymidine ([³H]-TdR) The amount of [³H]-TdR incorporated into the cellular DNA is quantified by liquid scintillation counting.

Human heparinized peripheral blood was obtained from healthy volunteers by cubital vein punction. The blood was diluted in PBS (1:3) and mononuclear cells were separated by centrifugation in Ficoll-Hypaque density gradient (Pharmacia, 1.077 g/ml) at 2200 rpm for 30 minutes. Following centrifugation, lymphocytes were washed in PBS and resuspended in cell culture medium (RMPI 1640, 2 mM glutamine, 100 U/ml penicillin, 100 μg/ml streptomycin, 10% fetal calf serum and sodium bicarbonate).

The cells were diluted at target density of 1100000 cells/ml and were added by pipette (180 μl) into 96/well microtiter plates. Four-fold dilutions of the intended test concentration were added at time zero in 20 μl aliquots to the microtiter plate wells. Usually, test compound was evaluated at six 4-fold dilutions. In routine testing, the highest well concentration was 266.7 μM. All drug concentrations were examined in duplicates. All wells with exception of unstimulated controls were activated with 50 μl of concanavalin A (25 μg/ml). Incubations of cells with test compounds lasted for 72 hours at 37° C., in 5% CO₂ atmosphere and 100% humidity. At the end of incubation period, the cells were assayed by using the [³H]-TdR:

Cell cultures were incubated with 0.5 μCi (20 μl of stock solution 500 μCi/ml) per well for 6 hours at 37° C. and 5% CO₂. The automated cell harvester was used to lyse cells in water and adsorb the DNA onto glass-fiber filters in the format of microtiter plate. The DNA incorporated [³H]-TdR was retained on the filter while unincorporated material passes through. The filters were dried at room temperature overnight, sealed into a sample bag with 10-12 ml of scintillant. The amount of [³H]-TdR present in each filter (in cpm) was determined by scintillation counting in a Betaplate liquid scintillation counter. The effective dose of immunosuppressant (ED) was calculated using the following equation: ED=(cpm_{drug exposed well}/mean cpm_{control wells})×100%. The EDs₅₀ value, the drug concentration inhibiting proliferation of 50% of lymphocytes, was calculated from the obtained dose response curves.

To evaluate immunosuppressive activity of substituted adenines, their ability to inhibit polyclonal mitogen induced proliferation of normal human lymphocytes was analyzed (Table 8). Our data demonstrate that the compounds have only marginal activity on ³H-thymidine incorporation, nonetheless, they efficiently inhibit proliferation of activated lymphocytes. Effective immunosuppressive dose of new derivatives under in vitro conditions was close to 1-20 μM.

TABLE 8
Immunosupressive activity of novel purine
derivatives.

| SUBSTITUENT | | | ED₅₀ |
|---|---|---|---|
| C2 | N6 | N9 | (μM) |
| (R)-2-hydroxypropylamino | [(R,S)-(1-phenyl-2-hydroxyethyl)amino] | isopropyl | 23 |
| (S)-2-hydroxypropylamino | [(R,S)-(1-phenyl-2-hydroxyethyl)amino] | isopropyl | 27 |
| Hexylamino | [(R,S)-(1-phenyl-2-hydroxyethyl)amino] | isopropyl | 56 |
| 2-hydroxyethylamino | [(R,S)-(1-phenyl-2-hydroxyethyl)amino] | isopropyl | 38 |
| (R)-1-(hydroxymethyl) propylamino | [(R,S)-(1-phenyl-2-hydroxyethyl)amino] | isopropyl | 12 |
| (S)-1-(hydroxymethyl) propylamino | [(R,S)-(1-phenyl-2-hydroxyethyl)amino] | isopropyl | 14 |
| (R)-2-hydroxypropylamino | [N-(2-(3,4-dihydroxyfenyl)ethyl)-N- | isopropyl | 7 |
| | methyl]amino | | |
| (S)-2-hydroxypropylamino | [N-(2-(3,4-dihydroxyfenyl)ethyl)-N- | isopropyl | 8 |
| | methyl]amino | | |
| Hexylamino | [N-(2-(3,4-dihydroxyfenyl)ethyl)-N- | isopropyl | 15 |
| | methyl]amino | | |
| 2-hydroxyethylamino | [N-(2-(3,4-dihydroxyfenyl)ethyl)-N- | isopropyl | 4 |
| | methyl]amino | | |
| (R)-1-(hydroxymethyl) propylamino | [N-(2-(3,4-dihydroxyfenyl)ethyl)-N- | isopropyl | 1.5 |
| | methyl]amino | | |
| (S)-1-(hydroxymethyl) propylamino | [N-(2-(3,4-dihydroxyfenyl)ethyl)-N- | isopropyl | 1.8 |
| | methyl]amino | | |
| 2-(1R-isopropyl-2- | [N-(2-(3,4-dihydroxyfenyl)ethyl)-N- | isopropyl | 0.54 |
| hydroxyethylamino) | methyl]amino | | |

### Example 13

Antiviral Activity

The activity of the compounds against HIV-1- and HIV-2-induced cytopathicity was examined in human lymphocyte MT-4 cells. The cells (300 000 cells/ml) were infected with 100 CCID₅₀ (1 CCID₅₀ is a virus quantity which causes cytopathicity effect in 50% of the cells under the experimental conditions) of HIV-1 or HIV-2 and added to 200 μl wells of a microtiter plate containing different dilutions of the tested-compounds. The infected cell cultures were incubated at 37° C. for 5 days in a humidified CO₂ incubator. The cytopathicity of the virus was examined by determination of MT-4 cell viability by trypan blue dye staining. The results are summarised in Table 9 with comparison on the prototype compounds.

Table 9 also shows the results of activity testing of novel compounds against MSV-induced transformation in murine embryo fibroblast C3H/3T3 cells. The cells were seeded in 1-ml-wells of 48-well plates and exposed to 80 PFU (plaque forming units) for 60-90 min. The virus was subsequently removed and culture medium containing appropriate concentrations of the tested compounds was added (1 ml per well). At day 6-post infection, MSV-induced transformation of the cell culture was examined microscopically. The results are summarised in Table 9 in comparison with the data on the prototype compounds.

TABLE 9
Anti-retroviral activity of novel compounds
substituted at R9 by PMP (N-(2-phosphonomethoxy-
propyl)group) or PME (N-(2-phosphonomethoxyethyl)-
derivative) (μg/ml) (R2 = NH₂). IC₅₀ values (μg/ml).

| | | HIV-1 | | HIV-2 | |
|---|---|---|---|---|---|
| R6 | MSV | MT-4 | CEM | MT-4 | CEM |

PME-derivatives

| | | | | | |
|---|---|---|---|---|---|
| Amino | 0.6 | 2.67 | 6.9 | ND | ND |
| Cyclohexylamino | 0.26 | 5.7 | >20 | 4.8 | >20 |
| Benzylamino | 1.5 | 50 | >20 | 49 | >20 |
| 3,4-dihydroxybenzylamino | 1.3 | 47 | >20 | 45 | >20 |
| [(R,S)-(1-phenyl-2-hydroxyethyl)amino] | 1.8 | 56 | >20 | 57 | >20 |
| [N-(2-(3,4-dihydroxyfenyl)ethyl)-N-methyl]amino | 0.9 | 45 | >20 | 32 | >20 |
| PMP-derivatives | | | | | |
| Cyclohexylamino | 3.78 | 3.4 | 4.5 | 5.8 | 8.5 |
| 3,4-dihydroxybenzylamino | 2.54 | 3.2 | 4.1 | 4.6 | 8.3 |
| [(R,S)-(1-phenyl-2-hydroxyethyl)amino] | 6.32 | 10.1 | >20 | 11.2 | >20 |
| [N-(2-(3,4-dihydroxyfenyl)ethyl)-N-methyl]amino | 1.37 | 2.1 | 5.2 | 3.2 | 7.8 |

The PMP (N-(2-phosphonomethoxypropyl) derivative) and PME (N-(2-phosphonomethoxyethyl) derivative) compounds of the formula I showed marked anti-HIV activity in vitro. HIV-1 and HIV-2 did not differ in sensitivity to the test compounds. (R)-PMP compounds were markedly inhibitory to retroviruses at 2-3 μg/ml and not toxic to the cells at 100 μg/ml. Its selectivity index (ratio cytotoxic dose/antivirally active dose) proved superior over that of the prototype compound PME. The (S)-enantiomer of PME was devoid of marked anti-retroviral activity. (R)-PMPD were exquisitely inhibitory to retrovirus replication (EC50 0.01-0.1 μg/ml) and not toxic to the cells at 100 μg/ml. It proved superior over PMEA and other prototype compounds in terms of both antiviral activity and lack of toxicity. Its selectivity index was higher than 2000 for HIV-1 and HIV-2.

### Example 14

Dry Capsules

5000 capsules, each of which contain 0.25 g of one of the purine derivatives of formula I, II and III, as defined above as active ingredient, are prepared as follows:

| Composition | | |
|---|---|---|
| Active ingredient | 1250 | g |
| Talc | 180 | g |
| Wheat starch | 120 | g |
| Magnesium stearate | 80 | g |
| Lactose | 20 | g |

Preparation Process

The powdered substances mentioned are pressed through a sieve of mesh width 0.6 mm. Portions of 0.33 g of the mixture are transferred to gelatine capsules with the aid of a capsule-filling machine.

### Example 15

Soft Capsules

5000 soft gelatine capsules, each of which contain 0.05 g of one of the purine derivatives of formula I, II and III as defined above as active ingredient, are prepared as follows:

| Composition | | |
|---|---|---|
| Active ingredient | 250 | g |
| Lauroglycol | 2 | litres |

Preparation Process

The powdered active ingredient is suspended in Lauroglykol® (propylene glycol laurate, Gattefossé S. A., Saint Priest, France) and ground in a wet-pulveriser to a particle size of about 1 to 3 μm. Portions of in each case 0.419 g of the mixture are then transferred to soft gelatine capsules by means of a capsule-filling machine.

### Example 16

Soft Capsules

5000 soft gelatine capsules, each of which contain 0.05 g of one of the purine derivatives of formula I, II or III defined above as active ingredient, are prepared as follows:

| Composition | | |
|---|---|---|
| Active ingredient | 250 | g |
| PEG 400 | 1 | litre |
| Tween 80 | 1 | litre |

Preparation Process

The powdered active ingredient is suspended in PEG 400 (polyethylene glycol of Mr between 380 and about 420, Sigma, Fluka, Aldrich, USA) and Tween® 80 (polyoxyethylene sorbitan monolaurate, Atlas Chem. Inc., Inc., USA, supplied by Sigma, Fluka, Aldrich, USA) and ground in a wet-pulveriser to a particle size of about 1 to 3 mm. Portions of in each case 0.43 g of the mixture are then transferred to soft gelatine capsules by means of a capsule-filling machine.

1. Purine derivatives and related aza-deaza analogues represented by general formula I: 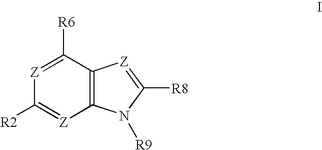
and pharmaceutically acceptable salts thereof, wherein:
Z is N or CH, provided that at most one Z is CH;
R6 is an (un)substituted adamamntyl or a di- or more substituted arylalkylamino group;
R8 is H, halogen, hydroxyl, amino, carboxyl, cyano, nitro, amido, sulfo, sulfamido, carbamino, (substituted) alkyl, (substituted) acyl, (substituted) cycloalkyl, (substituted) cycloheteralkyl, (substituted) arylalkyl, (substituted) heteroalkyl, (substituted) heteroaryl, (substituted) heterocycle, (substituted) heteroarylalkyl, (substituted) cycloalkyl alkyl, (substituted) aryl, (substituted) cycloheteroalkyl alkyl or R8′-X, wherein
X is is —NH—, —N(alkyl)-, —O— or —S—; and
R8′ is H, (substituted) alkyl, (substituted) acyl, amido, sulfo, (substituted) cycloalkyl, (substituted) aryl, (substituted) heterocycle, (substituted) heteroaryl, (substituted) arylalkyl, (substituted) cycloheteroalkyl, (substituted) heteroarylalkyl, (substituted) heteroalkyl, (substituted) cycloalkyl alkyl or (substitited) cycloheteroalkyl alkyl;
R2 is H, halogen, amido, carbamino, carboxyl, sulfamido, (subsituted) alkyl, (substituted) cycloalkyl, (substituted) cycloalkyl alkyl, (substituted) arylalkyl, (substituted) heteroalkyl, (substituted) heteroarylalkyl, (substituted) cycloheteroalkyl alkyl or R2′-X wherein
X is an —NH—, —N(alkyl)—, —O— or —S—;
R2′ is H, (substituted) alkyl, (substituted) acyl, amido, sulfo, carbamino, (substituted) cycloalkyl, (substituted) aryl, (substituted) heterocycle, (substituted) heteroaryl, (substituted) arylalkyl, (substituted) cycloheteroalkyl, (substituted) heteroarylalkyl, (substituted) heteroalkyl, (substituted) cycloalkyl alkyl or (substituted) cycloheteroalkyl alkyl; and
R9 is H, (substituted) alkyl, (substituted) acyl, carboxyl, amido, sulfo, sulfamido, carbamino, (substituted) cycloalkyl, (substituted) cycloalkyl alkyl, (substituted) cycloheteroalkyl alkyl, (substituted) cycloheteroalkyl, (substituted) aryl, (substituted) heterocycle, (substituted) heteroaryl, (substituted) arylalkyl, (substituted) heteroarylalkyl, (substituted) heteroalkyl; or —(CH₂)ₙ—R9′, wherein
n=1-2; and
R9′ is —X(CH₂)ₘY; wherein
X is —O—, —S—, —NH— or —N(alkyl)-;
m=1-2;
Y is carboxyl, amido, sulfo, sulfamido, hydroxy, alkoxy, mercapto, alkylmercapto, amino, alkylamino, carbamino —PO(OH)₂, —PO(Oalkyl)₂, —PO(NHalkyl)₂, —PO(Oalkyl)(NHalkyl), —PO(OH)(Oalkyl), —PO(OH)(NHalkyl); or
R9 is —(CH₂CHD)-R9′, wherein
R9′ is —X(CH₂)ₘY; wherein
X is —O—, —S—, —NH—, —N(alkyl)-;
m=1-2;
Y is carboxyl, amido, sulfo, sulfamido, hydroxy, alkoxy, mercapto, alkylmercapto, amino, alkylamino, carbamino, —PO(OH)₂, PO(Oalkyl)₂, —PO(NHalkyl), —PO(OH)(Oalkyl), —PO(OH)(NHalkyl), PO(Oalkyl)(Nalkyl); and
D is (substituted) alkyl;
wherein at least one of R2, R6, R8 or R9 is an amine substituted with a catechol group or related group.

2. Purine derivative as claimed in claim 1, wherein R6 is an amine catechol, an amine-linker catechol, or (R,S)-(1-phenyl-2-hydroxyethyl)amino.

3. Purine derivative as claimed in claim 1 or claim 2, wherein
R2 is H halogen
C₁-C₆ branched or linear, saturated or unsaturated lower alkyl such as methyl, ethyl, propyl, isopropyl, butyl, isobutyl, vinyl, allyl, ethinyl, propenyl, propinyl or isopenten-2-yl, optionally substituted with halogen, amino, hydroxy, mercapto, alkoxy, alkylamino, dialkylamino, alkylmercapto, carboxyl, amido, sulfo, sulfamido or carbamino;
C₃-C₅ cycloalkyl, such as cyclopropyl, cyclopentyl, cyclohexyl, or adamantyl;
cycloalkyl, optionally substituted with halogen, amino, hydroxy, mercapto, alkoxy, alkylamino, dialkylamino, alkylmercapto, carboxyl, amido, sulfo, sulfamido or carbamino;
cycloalkyl alkyl (—R-cycloalkyl), wherein R is a lower alkyl such as methyl, ethyl, propyl, isopropyl, vinyl, ethinyl, propenyl or propinyl;
arylalkyl (—R—Ar), wherein R is a lower alkyl such as methyl, ethyl, propyl, isopropyl, vinyl, propinyl, propenyl or ethinyl, and wherein Ar is phenyl, biphenyl, tetrahydronaphthyl, naphthyl, anthryl, indenyl or fenanthryl, optionally substituted with the groups as defined for cycloalkyl;
heteroalkyl (—R-Het), wherein R is a lower alkyl such as methyl, ethyl, propyl, isopropyl, vinyl, propinyl, propenyl or ethinyl, and Het is thienyl, furyl, pyranyl, pyrrolyl, imidazolyl, pyrazolyl, pyridyl, pyrazolinyl, pyrimidinyl, pyridazinyl, isothiazolyl or isoxazolyl, optionally substituted with substituents as defined for cycloalkyl;
heteroaryl alkyl (—R-HetAr), wherein R is a lower alkyl, such as methyl, ethyl, propyl, isopropyl, vinyl, propinyl, propenyl, or ethinyl, and HetAr is benzothienyl, naphthothienyl, benzofuranyl, chromenyl, indolyl, isoindolyl, indazolyl, quinolinyl, isoquinolinyl, phtalazinyl, quinaxalinyl, cinnolinyl or quinazolinyl;
cycloheteroalkyl (—R-cycloheteroalkyl), wherein R is a lower alkyl such as methyl, ethyl, propyl, isopropyl, vinyl, propinyl, propenyl or ethinyl, and wherein cycloheteroalkyl is pyrrolidinyl, piperidinyl, morfolinyl, imidazolidinyl, imidazolinyl or quinuclidinyl, optionally substituted with hydroxyl, amino, mercapto, carboxyl, amido or sulfo substituents; or
R2′-X, wherein
X is —NH—, —O—, —S— or —N(alkyl)-, wherein alkyl is methyl, ethyl, propyl, isopropyl, vinyl, ethinyl, allyl, propargyl or isopentenyl; and
R2′ is
H
C₁-C₆ branched or linear, saturated or unsaturated alkyl, such as methyl, ethyl, isopropyl, butyl, isobutyl, vinyl, allyl, propenyl, propargyl, propinyl, isopentenyl, or isobutenyl, optionally substituted by 1 to 3 substituents, such as halogen, amino, hydroxyl, mercapto, alkoxy, alkylmercapto, alkylamino, carboxyl, amido, sulfo, sulfamido or carbamino;
acyl (—C(O)R), wherein R is a branched or linear, saturated or unsaturated lower alkyl such as methyl, ethyl, propyl, isopropyl, butyl, isobutyl, vinyl, allyl, propenyl, propargyl, propinyl, isopentenyl or isobutenyl, optionally substituted by 1 to 3 substituents, such as halogen, amino, hydroxyl, mercapto, alkoxy, alkylmercapto, alkylamino, carboxyl, amido, sulfo, sulfamido or carbamino;
amido (—C(O)NRR′), wherein R and R′ independently are H, C₁C₆ branched or linear, saturated or unsaturated alkyl, and wherein R or R′ optionally are substituted by suitable substituents such as methyl, ethyl, propyl, isopropyl, butyl, isobutyl, vinyl, allyl, propenyl or propargyl;
sulfo (—SO₃R), wherein R is H, or a branched or linear, saturated or unsaturated C₁-C₆ alkyl, optionally substituted by halogen, amino, hydroxyl, mercapto, carboxyl, amido or carbamino;
carbamino (—NHC(O)R), wherein R is a branched or linear, saturated or unsaturated alkyl such as methyl, ethyl, propyl, isopropyl, allyl, propargyl, isopentenyl or isobutenyl, or R is hydroxyl, amino, alkoxy or alkylamino, and wherein R optionally is substituted with halogen, amino, hydroxyl, mercapto, carboxyl or amido;
C₃-C₁₅ cycloalkyl, such as cyclopropyl, cyclopentyl or cyclohexyl;
cycloalkyl, optionally substitued with 1 to 3 independent substituents, such as halogen (such as chloro or fluoro), amino, hydroxyl, mercapto, alkoxy (such as methoxy), alkylamino, dialkylamino, alkylmercapto, carboxyl, amido, sulfo, sulfamido, carbamino, nitro or cyano;
cycloalkyl alkyl (—R(cycloalkyl)), wherein R is a branched or linear, saturated or unsaturated lower alkyl such as methyl, ethyl, propyl, isopropyl, allyl, propargyl, isopentenyl or isobutenyl, and cycloalkyl is as defined for cycloalkyl and substituted cycloalkyl;
aryl, such as phenyl, biphenyl, naphthyl, tetrahydronaphthyl, fluorenyl, indenyl or fenanthrenyl, optionally substituted by 1 to 3 substituents such as defined for substituted cycloalkyl;
heterocycle such as thienyl, furyl, pyranyl, pyrrolyl, imidazolyl, pyrazolyl, pyridyl, pyrazinyl, pyrimidinyl, pyridazinyl, isothiazolyl or isoxazyl, optionally substituted by 1 to 2 substituents, such as defined for substituted cycloalkyl;
heteroalkyl (—R-Het), wherein R is a lower alkyl such as methyl, ethyl, propyl, isopropyl, vinyl, propinyl, propenyl or ethinyl, and Het is as defined for the heterocycle group, optionally substituted by 1 to 2 substituents as defined for substituted cycloalkyl;
heteroaryl (—R-HetAr), wherein R is methyl, ethyl, propyl, isopropyl, vinyl, propinyl or propenyl, and HetAr is benzothienyl, naphthothienyl, benzofuranyl, chromenyl, indolyl, isoindolyl, indazolyl, quinolinyl, isoquinolinyl, phtalazinyl, quinaxalinyl, cinnolinyl or quinazolinyl;
arylalkyl (—RAr), wherein R is a branched or linear, saturated or unsaturated C₁-C₆ lower alkyl such as methyl, ethyl, propyl, isopropyl, vinyl, propinyl or propenyl, and the aryl ring(s) optionally are substituted by 1 to 3 independent substituents as defined for substituted cycloalkyl; —cycloheteroalkyl such as piperidinyl, piperazinyl, morfolinyl, pyrrolidinyl or imidazolidinyl, optionally substituted by 1 to 2 substituents such as those defined for substituted cycloalkyl; cycloheteroalkyl alkyl (—R(cycloheteroalkyl)), wherein R is as defined for arylalkyl, and the cycloheteroalkyl ring optionally is substituted with 1 to 2 groups as defined for cycloalkyl;
heteroarylalkyl (—R-HetAr_, wherein R is a branched or linear, saturated or unsaturated lower alkyl, such as methyl, ethyl, propyl, isopropyl, vinyl, propinyl, propenyl, allyl, propargyl or isopentenyl, and HetAr is benzothienyl, benzofuranyl, chromenyl, indolyl, isoindolyl, indazolyl, quinolinyl, phthalazinyl, quinoxalinyl, quinazolinyl, carbazolyl, acridinyl, indolinyl or isoindolinyl, and wherein R and HetAr optionally independently are substituted by halogen, hydroxyl, amino, mercapto, carboxyl or amido;
R8 is H, halogen, hydroxyl, amino, carboxyl, cyano, nitro, amido, sulfo, sulfamido, carbamino; or (substituted) alkyl, acyl, (substituted) cycloalkyl, (substituted) cycloheteroalkyl, cycloalkyl alkyl, (substituted) aryl, arylakyl, heterocycle, (substituted) heteroaryl, heteroalkyl or heteroarylalkyl, wherein these groups are as defined for R2; or
R8′-X, wherein
X is —NH—, —O—, —S— or —N(alkyl)-, wherein alkyl is C₁-C₆ alkyl, methyl, ethyl, propyl, isopropyl, vinyl, allyl or propargyl; and
R8′ is (substituted) alkyl, acyl, amido, (substituted) cycloalkyl, (substituted) cycloheteroalkyl, cycloalkyl alkyl, (substituted) aryl, arylakyl, heterocycle, (substituted) heteroaryl, heteroalkyl or heteroarylalkyl, wherein these groups are as defined for R2′; and
R9 is H, (substituted) alkyl, acyl, amido, carboxyl, sulfo, carbamino, (substituted) cycloalkyl, (substituted) cycloheteroalkyl, cycloalkyl alkyl, (substituted) aryl, arylalkyl, heterocycle, (substituted) heteroaryl, heteroalkyl or heteroarylalkyl, and wherein these groups are as defined for R2,
or (CH₂)ₙ—R9′, wherein
n=1-2;
R9′ is —X(CH₂)Y; wherein
X is —O—, —S—, —NH— or —N(alkyl)-, wherein alkyl is a linear or branched, saturated or unsaturated C₁-C₆ alkyl, such as methyl, ethyl, propyl, isopropyl, vinyl, allyl or propargyl;
m=1-2;
Y is hydroxy, mercapto, amino, alkoxy, alkylmercapto, alkylamino, carboxyl, sulfo, sulfamido, carbamino, —PO(OH)₂, —PO(Oalkyl)(OH), —PO(Oalkyl)₂, —PO(Oalkyl)(NHalkyl), —PO(NHalkyl)₂, —PO(NHalkyl)(OH);
or (CH₂CHD)-R9′, wherein
R9′ is —X(CH₂)ₘY; wherein
X is —O—, —S—, —NH— or —N(alkyl)-, wherein alkyl is a linear or branched, saturated or unsaturated C₁-C₆ alkyl, such as methyl, ethyl, propyl, isopropyl, vinyl, allyl or propargyl;
m=1-2;
Y is hydroxy, mercapto, amino, alkoxy, alkylmercapto, alkylamino, carboxyl, sulfo, sulfamido, carbamino, —PO(OH)₂, —PO(Oalkyl)(OH), —PO(Oalkyl)₂, —PO(Oalkyl)(NHalkyl), —PO(NHalkyl)₂, or PO(NHalkyl)(OH); and
D is a lower alkyl, optionally substituted by Y.

4. Purine derivatives as claimed in claims any of claims 1-3, wherein the purine derivatives are chosen from the group consisting of 6-(3,4-dihydroxybenzyl)-aminopurine, 6-(3,4-dihydroxybenzyl)amino-9-isopropyl-purine, 2-(1-hydroxymethylpropylamino)-6-(3,4-dihydroxy-benzyl)amino-9-isopropylpurine, 2-(R)-(2-hydroxymethyl-pyrrolidine-1-yl)-6-(3,4-dihydroxybenzyl)amino-9-ispopropylpurine, 2-(2-aminopropylamino)-6-(3,4-dihydroxybenzyl)amino-9-isopropylpurine, 2-(2-hydroxy-propylamino)-6-(3,4-dihydroxybenzyl)amino-9-isopropyl-purine, 2-(R)-(1-isopropyl-2-hydroxyethylamino)-6-(3,4-dihydroxybenzyl)amino-9-isopropylpurine, 6-[N-(3,4-dihydroxybenzyl)-N-methyl]amino-9-isopropylpurine, 6-[N-(3,4-dihydroxybenzyl)-N-methyl]aminopurine, 6-[N-(3,4-dihydroxybenzyl)-N-methyl]amino-8-fluoropurine, 6-[N-(3,4-dihydroxybenzyl)-N-methyl]amino-9-isopropylpurine, 2-(2-hydroxypropylamino)-6-[N-(3,4-dihydroxybenzyl)-N-methyl]amino-9-isopropylpurine, 2-(R)-(1-isopropyl-2-hydroxyethylamino-6-[N-(3,4-dihydroxybenzyl)-N-methyl]amino-9-isopropylpurine, 6-[1-(3,4-dihydroxy-phenyl)ethyl]amino-9-isopropylpurine, 6-[1-(3,4-dihydroxyphenyl)ethyl]aminopurine, 6-[1-(3,4-dihydroxy-phenyl)ethyl]amino-8-fluoropurine, 6-[1-(3,4-dihydroxy-phenyl)ethyl]amino-9-isopropylpurine, 2-(2-hydroxy-propylamino)-6-[1-(3,4-dihydroxyphenyl)ethyl]amino-9-isopropylpurine, 2-(R)-(1-isopropyl-2-hydroxyethylamino-6-[1-(3,4-dihydroxyphenyl)ethyl]amino-9-isopropylpurine, 2-(R)-(1-isopropyl-2-hydroxyethylamino-6-[N-(2-(3,4-dihydroxyfenyl)ethyl)-N-methyl]amino-9-isopropylpurine, 6-[N-(2-(3,4-dihydroxyfenyl)ethyl)-N-methyl]amino-9-isopropylpurine, 6-[N-(2-(3,4-dihydroxyfenyl)ethyl)-N-methyl]amino-8-bromo-9-isopropylpurine, 6-[N-(2-(3,4-dihydroxyfenyl)ethyl)-N-methyl]aminopurine, 6-(R,S)-hydroxy-1-phenylethylamino-9-isopropylpurine, 6-[(R,S)-(1-phenyl-2-hydroxyethyl)aminopurine, 2-(1R-isopropyl-2-hydroxyethylamino)-6-[(R)-(1-phenyl-2-hydroxyethyl)amino]-9-isopropylpurine, 2-(R)-(1-isopropyl-2-hydroxyethylamino-6-[(R,S)-(1-phenyl-2-hydroxyethyl)amino]-isopropylpurine, 2-chloro-6-[(R,S)-(1-phenyl-2-hydroxyethyl)amino]-9-isopropylpurine, 2-chloro-6-[(R,S)-(1-phenyl-2-hydroxyethyl)amino]purine, 6-[(R,S)-(1-phenyl-2-hydroxyethyl)-9-(R)-(2-phosphono-methoxypropyl)purine, 6-[N-(3,4-dihydroxybenzyl)-N-methyl]amino-9-(R)-(2-phosphonomethoxypropyl)purine, 2-Amino-6-[(R,S)-(1-phenyl-2-hydroxyethyl)-9-(R)-(2-phosphonomethoxypropyl)purine, 2-Amino-6-[N-(3,4-dihydroxybenzyl)-N-methyl]amino-9-(R)-(2-phosphonomethoxypropyl)purine, 2-Amino-6-(3,4-dihydroxybenzyl)-amino-9-(R)-(2-phosphonomethoxypropyl)purine, and 2-Amino-6-[N-(2-((3,4-dihydroxyfenyl)ethyl)-N-methyl]amino-9-(R)-(2-phosphonomethoxypropyl)purine.

5. Method to prepare 6-substituted purine derivatives of formula I in claim 1, wherein R6 substituents are as defined in claim 1-2, wherein 6-chloropurine is dissolved in n-butanol and reacted with an appropriate amine and excess triethylamine.

6. Method to prepare 6,9-disubstituted purine derivatives of formula I in claim 1, wherein R6, R9 substituents are as defined in claim 1-3, wherein 6-substituted purine derivatives in DMSO, or DMF are reacted with powdered calcium carbonate followed by R⁹-halogen.

7. Method to prepare 6, 8, 9-trisubstituted purine derivatives of formula I in claim 1, wherein R6, R8, R9 substituents are as defined in claims 1-3 from 6,9-disubstituted purines by S, bromination (Br₂, CHCl₃−20° C.) and subsequent S_{N} displacement of C⁸—Br by nucleophiles.

8. Method to prepare 2,6-disubstituted purine derivatives of formula I in claim 1, wherein R2, R6 substituents are as defined in claims 1-3, wherein 2,6-dichloropurine is reacted with an appropriate nucleophile and substitution of C₂-C₁ is achieved by reaction with a second nucleophile at a temperature 160-180° C.

9. Method to prepare 2,6,9-trisubstituted purine derivatives of formula I in claim 1, wherein R2, R6, R9 substituents are as defined in claims 1-3, wherein 2-chloro-6-substituted purine derivatives are alkylated and subsequent reacted with R²—SH or R²—NH.

10. Method to prepare 2,9-disubstituted purine derivatives of formula I in claim 1, wherein R2, R9 substituents are as defined in claims 1-3, wherein powdered potassium carbonate is added to 2,6-dichloropurine in DMSO or DMF, followed by addition of R⁹-halogen, whereafter 2-chloro-9-alkylpurine is provided by selective hydrogenolysis of C⁶—Cl, and the last nucleophilic substitution of C²—Cl is achieved by reaction with an appropriate nucleophile at a temperature 140-180° C.

11. Method to prepare purine derivatives of formula I in claim 1, wherein R2, R6, R8 substituents are as defined in claims 1-3, wherein 2,6-disubstituted purine derivatives are brominated to get 2,6,8-trisubstituted purine derivatives whereafter substitution of C⁸Br in the 2,6-disubstituted-8-bromo-purines is achieved by reaction with excess of nucleophile at a temperature 160-180° C.

12. Method to prepare purine derivatives of claim 1-4 wherein R2, R6, and R9 are as defined in claims 1-3, wherein 2,6-dichloropurines are alkylated.

13. Purine derivatives as claimed in any of claims 1-4 for use as an inhibitor of cyclin-dependent kinase proteins.

14. Purine derivatives as claimed in any of claims 1-4 for use as an antiviral, antimitotic, antiproliferative, immunomodulating, immune-suppressive, anti-inflammatory, antimicrobial and/or antitumor agent.

15. Purine derivatives as claimed in any of claims 1-4 for use as a modulator of α, β-adrenergic and/or purinergic receptors.

16. Purine derivatives as claimed in any of claims 1-4 for use as an inhibitor of proliferation of hematopoietic cells and cancer cells.

17. Purine derivatives as claimed in any of claims 1-4 for use as an inducer of apoptosis in cancer cells.

18. Purine derivatives as claimed in any of claims 1-4 and 13-17 for use in treatment of the human or animal body.

19. Pharmaceutical composition comprising one or more purine derivatives as claimed in any of claims 1-4 and 13-18 and a pharmaceutically acceptable carrier or diluent.

20. Use of purine derivatives as claimed in claims 1-4 and 13-18 for the preparation of affinity absorption matrices.

21. Method for inhibiting cell proliferation in mammals comprising administering an effective amount of one or more purine derivatives as claimed in claims 1-4 and 13-18 to the mammal together with a pharmaceutical acceptable carrier.

22. Method for treatment of viral infections in mammals comprising administering an effective amount of one or more purine derivatives as claimed in claims 1-4 and 13-18 to the mammal together with a pharmaceutical acceptable carrier.

23. Method for treatment of cancer in mammals comprising administering an effective amount of one or more purine derivatives as claimed in claims 1-4 and 13-18 to the mammal together with a pharmaceutical acceptable carrier, optionally in combination with one or more cytostatic agents.

24. Method to suppress immune stimulation in mammals comprising administering an effective amount of one or more purine derivatives as claimed in claims 1-4 and 13-18 to the mammal together with a pharmaceutical acceptable carrier.
